(19) United States
(12) Patent Application Publication
(10) Pub. No.: US 2020/0276209 A1
(43) Pub. Date: Sep. 3, 2020

(54) METHODS OF TREATING EPILEPSY OR STATUS EPILEPTICUS

(71) Applicant: Sage Therapeutics, Inc., Cambridge, MA (US)

(72) Inventors: Helen Colquhoun, Arlington, MA (US); Stephen Jay Kanes, Brynlawn, PA (US)

(21) Appl. No.: 16/645,975

(22) PCT Filed: Sep. 11, 2018
(86) PCT No.: PCT/US2018/050444

§ 371 Date: Mar. 10, 2020

Related U.S. Application Data
(60) Provisional application No. 62/557,622, filed on Sep. 12, 2017.
(60) Provisional application No. 62/557,025, filed on Sep. 11, 2017.

(51) Int. Cl. A61K 31/57; A61K 47/40; A61P 25/08

(52) U.S. Cl. CPC A61K 31/57; A61K 9/0019; A61P 25/08; A61K 47/40

(57) ABSTRACT

Described herein are methods of treating epilepsy or status epilepticus, e.g., convulsive status epilepticus, e.g., early status epilepticus, established status epilepticus, refractory status epilepticus, super-refractory status epilepticus, e.g., super-refractory generalized status epilepticus; non-convulsive status epilepticus, e.g., generalized status epilepticus, complex partial status epilepticus; generalized periodic epileptiform discharges; periodic lateralized epileptiform discharges; a seizure, e.g., acute repetitive seizures, cluster seizures, the method comprising administering to the subject allopregnanlone.

### CROSS-REFERENCE TO RELATED APPLICATIONS

This application claims priority to U.S. Ser. No. 62/557,025 filed Sep. 11, 2017 and U.S. Ser. No. 62/557,622 filed Sep. 12, 2017, the contents of which are incorporated herein by reference.

### FIELD OF THE INVENTION

The present invention generally relates to methods of treating a seizure-related disorder such as epilepsy or status epilepticus by administering allopregnanolone.

### SUMMARY OF THE INVENTION

Described herein are methods of treating a seizure-related disorder such as epilepsy or status epilepticus. e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; periodic lateralized epileptiform discharges; or a seizure, e.g., acute repetitive seizures or cluster seizures, to a subject in need, the methods comprising administering to the subject allopregnanolone.

In one aspect, provided herein is a method of treating a subject having a seizure-related disorder, wherein said seizure-related disorder is preceded by a condition related to a structural modification in the brain of said subject, the method comprising: administering to said subject, an effective amount of allopregnanolone, thereby treating said subject. In some embodiments, the structural modification is a lesion in the brain that is visible using standard imaging techniques, e.g., magnetic resonance imaging (MRI) or a computerized tomography (CT) scan. In some embodiments, the epilepsy or status epilepticus is related to, e.g., caused by, the condition related to a structural modification in the brain of said subject. In some embodiments, the structural modification is a vascular structural modification. In some embodiments, the condition associated with the structural modification is a tumor (e.g., the tumor is a source of the structural modification). In some embodiments, the condition related to a structural modification is a brain aneurysm with associated hemorrhage. In some embodiments, the condition related to a structural modification is a intraparenchymal hemorrhage. In some embodiments, the condition related to a structural modification is a brain arteriovenous malformation with associated hemorrhage. In some embodiments, the condition related to a structural modification is an ischemic stroke. In some embodiments, the condition related to a structural modification is a focal cortical dysplasias or malformation of cortical development. In some embodiments, the condition related to a structural modification is an intraparenchymal brain tumor. In some embodiments, the condition related to a structural modification is post-traumatic porenenchephaly or gliosis. In some embodiments, the condition related to a structural modification is a cerebral abscess. In some embodiments, the condition related to a structural modification is a central nervous system infection, e.g., neurocysticercosis. In some embodiments, the condition related to a structural modification is encephalitis. In some embodiments, the condition related to a structural modification is multiple sclerosis. In some embodiments, the condition related to a structural modification is a demyelinating lesion.

In another aspect, the provided herein a method of treating a subject having a seizure-related disorder, wherein said seizure-related disorder is preceded by a condition selected from the group consisting of a brain aneurysm with associated hemorrhage, an intraparenchymal hemorrhage, a brain arteriovenous malformation with associated hemorrhage, an ischemic stroke, a focal cortical dysplasias or malformation of cortical development, an intraparenchymal brain tumor, post-traumatic porenenchephaly or gliosis, a cerebral abscess, a central nervous system infection, encephalitis, multiple sclerosis, and a demyelinating lesion, the method comprising: administering to said subject, an effective amount of allopregnanolone, thereby treating said subject.

In some embodiments of any of the foregoing, the invention features a method of treating a subject having epilepsy or status epilepticus by administering in combination to the subject allopregnanolone and a benzodiazepine, In some embodiments, the method further comprises administering at least one of the allopregnanolone or benzodiazepine parenterally (e.g., intranasally, buccally, intravenously or intramuscularly, for example, intravenously or intramuscularly). In some embodiments, both the allopregnanolone and benzodiazepine are administered parenterally.

In some embodiments, the allopregnanolone and benzodiazepine are co-administered (e.g., administered simultaneously). In some embodiments, the allopregnanolone and benzodiazepine are administered sequentially. In some embodiments, allopregnanolone and benzodiazepine are administered in a single dosage form.

When the agents described herein (e.g., the allopregnanolone and a benzodiazepine) are administered in combination, both of the agents should be present at dosage levels of between about 1 to 100%, and more preferably between about 5 to 95% of the dosage normally administered in a in the absence of the combination regimen. The agents may be administered separately, as part of a multiple dose regimen. Alternatively, the agents may be part of a single dosage form, mixed together with the compounds of this invention in a single composition.

In some embodiments, the allopregnanlone is formulated for parenteral administration (e.g., intranasally, buccally, intravenously or intramuscularly, for example, intravenously or intramuscularly).

In some embodiments, the allopregnanolone is administered in a composition comprising a cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, complex. aβ-cyclodextrin, e.g., a sulfo butyl ether β-cyclodextrin, e.g., a β-cyclodextrin, e.g., a sulfo butyl ether β-cyclodextrin, e.g., CAPTISOL®.

In some embodiments, the cyclodextrin is a β-cyclodextrin. In an embodiment, the cyclodextrin is a sulfo butyl ether β-cyclodextrin. In an embodiment, the cyclodextrin is CAPTISOL®. In some embodiments, the cyclodextrin is a β-cyclodextrin disclosed in U.S. Pat. Nos. 5,874,418; 6,046,177; or 7,635,733, which are herein incorporated by reference.

In some embodiments, the allopregnanolone is a progestin derivative, and the cyclodextrin is a β-cyclodextrin. In an embodiment, the allopregnanolone is allopregnanolone and the cyclodextrin is CAPTISOL®.

In some embodiments, the allopregnanolone and cyclodextrin, e.g., a β-cyclodextrin, e.g., a sulfo butyl ether β-cyclodextrin, e.g., CAPTISOL®, complex is formulated for parenteral administration. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition. In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration between 0.25-30 mg/mL, 0.5-30 mg/mL; 1-30 mg/mL; 5-30 mg/mL, 10-30 mg/mL; 15-30 mg/mL, 0.25-20 mg/mL; 0.5-20 mg/mL; 1-20 mg/mL, 0.5-20 mg/mL; 1-20 mg/mL, 5-20 mg/mL, 10-20 mg/mL, 0.25-15 mg/mL, 0.5-15 mg/mL; 0.5-10 mg/mL; 1-15 mg/mL, 1-10 mg/mL; 1-5 mg/mL; 5-15 mg/mL; 5-10 mg/mL; 10-15 mg/mL; 1-10 mg/mL; 2-8 mg/mL; 2-7 mg/mL; 3-5 mg/mL; 5-15 mg/mL; 7-12 mg/mL; 7-10 mg/mL; 8-9 mg/mL; 3-5 mg/mL; or 3-4 mg/mL. In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 0.25 mg/mL, 0.5 mg/mL; 1.0 mg/mL; 1.5 mg/mL; 2.0 mg/mL; 2.5 mg/mL; 3.0 mg/mL; 3.5 mg/mL; 4.0 mg/mL; 4.5 mg/mL; 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10 mg/mL, 15 mg/mL, 20 mg/mL, 25 mg.mL, or 30 mg/mL. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 1.5 mg/mL. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 5 mg/mL. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 15 mg/mL.

In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising the cyclodextrin, e.g., a β-cyclodextrin, e.g., a sulfo butyl ether β-cyclodextrin, e.g., CAPTISOL®, at a concentration between 25-400 mg/mL; 25-300 mg/mL; 25-200 mg/mL; 25-100 mg/mL; 25-50 mg/mL; 50-400 mg/mL; 50-300 mg/mL; 60-400 mg/mL; 60-300 mg/mL; 150-400 mg/mL; 150-300 mg/mL; 200-300 mg/mL; 200-400 mg/mL; 30-100 mg/mL; 300-400 mg/mL; 30-100 mg/mL; 45-75 mg/mL; 50-70 mg/mL; 55-65 mg/mL; or 50-60 mg/mL. In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising the cyclodextrin, e.g., a β-cyclodextrin, e.g., a sulfo butyl ether β-cyclodextrin, e.g., CAPTISOL®, at a concentration of 25 mg/mL; 30 mg/mL; 35 mg/mL; 40 mg/mL; 45 mg/mL; 50 mg/mL; 55 mg/mL; 60 mg/mL; 65 mg/mL; 70 mg/mL; 75 mg/mL; 80 mg/mL; 85 mg/mL; 90 mg/mL, 95 mg/mL; 100 mg/mL; 150 mg/mL; 200 mg/mL; 250 mg/mL; 300 mg/mL; 350 mg/mL; or 400 mg/mL. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the cyclodextrin, e.g., a β-cyclodextrin, e.g., a sulfo butyl ether β-cyclodextrin, e.g., CAPTISOL®, at a concentration of 60 mg/ml. In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising between 2.5-40%, 2.5-30%, 2.5-20%, 2.5-10%, 5-40%, 5-30%, 5-20%, 5-10%, 6-40%, 6-30%, 6-20%, 6-10%, 10-40%, 10-30%, 10-20%, 20-40%, 20-30%, 25-40%, 25-30%, 3-10%, 4.5-7.5%, 5-7%, 5.5-6.5% of the cyclodextrin, e.g., CAPTISOL®. In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising 2.5%, 3%, 4%, 4.5%, 5%, 5.5%, 6%, 6.5%, 7%, 7.5%, 8%, 8.5%, 9%, 9.5%, 10%, 15%, 20%, 25%, 30%, 35% or 40% of the cyclodextrin, e.g., CAPTISOL®. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising 6% of the cyclodextrin. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising 15% of the cyclodextrin. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising 30% of the cyclodextrin.

In some embodiments, the allopregnanolone and CAPTISOL® complex is formulated as an aqueous composition with a pH between 3-10, 4-9, 4-8, 4-7, 4-6, 4-5, 5-9, 5-8, 5-7, 5-6, 4.5-7.5, or 5.5-7.5. In some embodiments, the allopregnanolone and CAPTISOL® complex is formulated as an aqueous composition with a pH about 3, 3.5, 4, 4.5, 5, 5.5, 6, 6.5, 7, 7.5, 8, 8.5, or 9. In an embodiment, the allopregnanolone and CAPTISOL® complex is formulated as an aqueous composition with a pH about 6.

In an embodiment, a composition comprising allopregnanoloneand cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, complex, comprises less than 100 ppm of a phosphate, and the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, has an absorption of less than 0.2 A.U. due to a drug-degrading agent, as determined by UV/vis spectrophotometry at a wavelength of 245 nm to 270 nm for an aqueous solution comprising 300 mg of the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, per mL of solution in a cell having a 1 cm path length.

In some embodiments, the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, has an absorption of less than 0.2 A.U. due to a color forming agent, as determined by UV/vis spectrophotometry at a wavelength of 320 nm to 350 nm for an aqueous solution comprising 500 mg of the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, per mL of solution in a cell having a 1 cm path length.

In some embodiments, the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, further comprises: less than 20 ppm of a sulfoalkylating agent; less than 0.5% wt. of an underivatized cyclodextrin; less than 1% wt. of an alkali metal halide salt; and less than 0.25% wt. of a hydrolyzed sulfoalkylating agent.

In some embodiments, the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, has an absorption of less than 0.2 A.U. due to a drug-degrading agent, as determined by UV/vis spectrophotometry at a wavelength of 245 nm to 270 nm for an aqueous solution comprising 500 mg of the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, per mL of solution in a cell having a 1 cm path length.

In some embodiments, the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, further comprises: less than 50 ppm of a phosphate; less than 10 ppm of a sulfoalkylating agent; less than 0.2% wt. of an underivatized cyclodextrin; less than 0.5% wt. of an alkali metal halide salt; and less than 0.1% wt. of a hydrolyzed sulfoalkylating agent; and wherein the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, has an absorption of less than 0.2 A.U. due to the color-forming agent, as determined by U/vis spectrophotometry at a wavelength of 320 nm to 350 nm for an aqueous solution comprising 500 mg of the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, per mL of solution in a cell having a 1 cm path length.

In some embodiments, the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, further comprises: less than 10 ppm of a phosphate; less than 2 ppm of a sulfoalkylating agent; less than 0.1% wt. of an underivatized cyclodextrin; less than 0.2% wt. of an alkali metal halide salt; and less than 0.08% wt. of a hydrolyzed sulfoalkylating agent; and wherein the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, has an absorption of less than 0.1 A.U. due to the color-forming agent, as determined by UV/vis spectrophotometry at a wavelength of 320 nm to 350 nm for an aqueous solution comprising 500 mg of the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, per mL of solution in a cell having a 1 cm path length.

In some embodiments, the cyclodextrin, e.g., a β-cyclodextrin, e.g., a sulfo butyl ether β-cyclodextrin, e.g., a β-cyclodextrin, e.g., a sulfo butyl ether β-cyclodextrin, e.g., CAPTISOL®, further comprises: less than 5 ppm of a phosphate; less than 0.1% wt. of an alkali metal halide salt; and less than 0.05% wt. of a hydrolyzed sulfoalkylating agent.

In some embodiments, the allopregnanolone and CAPTISOL® complex is formulated as an aqueous composition and is administered within 10 hours, 8 hours, 5 hours, 3 hours, 1 hour, or 0.5 hour after a seizure, e.g., a status epileptic seizure, e.g., a refractory status epileptic seizure has started. In some embodiments, the allopregnanolone and CAPTISOL® complex is formulated as an aqueous composition and is administered within 60 minutes, 45 minutes, 30 minutes, 15 minutes, 10 minutes, or 5 minutes after a seizure, e.g., a status epileptic seizure, e.g., a refractory status epileptic seizure has started. In some embodiments, the allopregnanolone and CAPTISOL® complex is formulated as an aqueous composition and is administered after a seizure, e.g., a status epileptic seizure, e.g., a refractory status epileptic seizure has lasted 5 minutes, 10 minutes, 15 minutes, 20 minutes, 30 minutes or 60 minutes.

In some embodiments, the the allopregnanolone and CAPTISOL® complex is formulated as an aqueous composition and is administered prior to the onset of a seizure, e.g., a status epileptic seizure, e.g., a refractory status epileptic seizure.

In some embodiments, the benzodiazepine is clonazepam, lorazepam, midazolam, or diazepam.

In some embodiments, the benzodiazepine is formulated for oral delivery. In some embodiments, the benzodiazepine is formulated for parenteral delivery (e.g., intranasally, buccally, intravenously or intramuscularly, for example, intravenously or intramuscularly).

In some embodiments, both the allopregnanolone and the benzodiazepine are formulated for parenteral delivery (e.g., intranasally, buccally, intravenously or intramuscularly, for example, intravenously or intramuscularly).

In some embodiments, the allopregnanolone and benzodiazepine, when administered in combination, are administered in an amount sufficient to achieve burst suppression (e.g., a predetermined burst suppression pattern, e.g., inter-burst intervals of between 2-30 seconds; as measured by a method of neurophysiological monitoring, e.g., EEG, CFM). In some embodiments, the allopregnanolone and benzodiazepine, when administered in combination is administered at a dose sufficient to achieve a predetermined burst suppression pattern, e.g., inter-burst intervals of between 2-30 seconds, 5-30 seconds, 10-30 seconds, 15-30 seconds, 1-30 seconds, 0-30 seconds, 2-20 seconds, 2-10 seconds, 5-20 seconds, 10-20 seconds, 15-25 seconds, 5-15 seconds or 5-10 seconds; as measured by a method of neurophysiological monitoring, e.g., EEG, CFM.

In one aspect, the invention features a method of treating a subject (e.g., human subject) having a seizure-related disorder, e.g., status epilepticus (SE), e.g., refractory status epilepticus (RSE) or super-refractory status epilepticus (SRSE), comprising: administering to said subject (e.g., human subject), an effective amount of allopregnanolone, wherein, concurrent with said administering, said subject (e.g., human subject) is under general anesthesia, thereby treating said subject (e.g., human subject).

In one aspect, the invention features a method of treating a subject (e.g., human subject) having a seizure-related disorder, e.g., status epilepticus (SE), e.g., refractory status epilepticus (RSE) or super-refractory status epilepticus (SRSE), comprising administering a first dose, e.g., a load dose, of allopregnanolone, e.g., to a patient under general anesthesia; administering a second dose, e.g., maintenance dose, of allopregnanolone, which is lower than said first dose; and administering a third dose, e.g., a downward taper dose, of allopregnanolone, said allopregnanolone doses being sufficient to treat said subject (e.g., human subject).

In some embodiments, said subject (e.g., human subject) is not under general anesthesia for at least a portion of the second dose. In some embodiments, said subject (e.g., human subject) is not under general anesthesia for at least a portion of the third dose. In some embodiments, said subject (e.g., human subject) is under general anesthesia during the administration of the first dose and during administration of a portion of the second dose, e.g., for at least, or up to, 6, 12, 24, or 47 hours of the second dose.

In some embodiments, the second dose is administered over a period of time that is at least 60, 65, 70, 80, 90, 100, 110, 120 times longer in duration than that of said first dose. In some embodiments, the second dose is administered over a period of time that not more than 80, 90, 100, 110, 120, 130, or 140 times longer in duration than that of said first dose.

In some embodiments, the second dose is administered over a period of time that at least 2, 3, 4, 5, 6 times longer in duration than that of said third dose. In some embodiments, the second dose is administered over a period of time that not more than 5, 6, 7, 8, 9, or 10 times longer in duration than that of said third dose.

In some embodiments, the infusion rate, e.g., amount of allopregnanolone delivered/unit time in the second dose, e.g., as measured in μg/kg/hour, is at least 2, 3, 4, 5, or 6 times lower than that of the first dose.

In some embodiments, one, two or all of said doses are injected, e.g., IV administrations. In some embodiments, said subject (e.g., human subject) has failed to respond to a first line treatment, e.g., a benzodiazepine (e.g. midazolam), e.g., as evidenced by a failure to induce an EEG pattern of burst suppression, failure to control seizure, continued seizure activity on EEG recording after 24 hours or more on the first line treatment, or failure to wean from the first line treatment without resuming seizure activity as evidenced by EEG recording.

In some embodiments, said subject (e.g., human subject) has failed to respond to a second line treatment, e.g., phenytoin, fos-phenytoin, valproate, phenobarbitol, or levetiracetam, e.g., as evidenced by a failure to induce an EEG pattern of burst suppression, failure to control seizure, continued seizure activity on EEG recording after 24 hours or more on the first line treatment, or failure to wean from the first line treatment without resuming seizure activity as evidenced by EEG recording.

In some embodiments, the method further comprises administering an amount of an aesthetic effective to place said subject (e.g., human subject) under general anesthesia. In some embodiments, said anesthetic is selected from a benzodiazepine (e.g. midazolam), propofol, and pentobarbital.

In some embodiments, the method further comprises a weaning period in which said subject (e.g., human subject) is weaned from said general anesthesia. In some embodiments, said weaning period is initiated during the administration of said second dose. In some embodiments, said weaning period is completed during the administration of said second dose. In some embodiments, said weaning period is initiated within 12, 24, 36, 48, 60 or 72 hours after initiation or completion of the first dose of allopregnanolone. In some embodiments, said weaning period is initiated at 48 hours after initiation or completion of the first dose of allopregnanolone. In some embodiments, said weaning period is 18 to 30 hours, 20 to 28 hours, or 22 to 26 hours in duration. In some embodiments, said weaning period is 24 hours in duration.

In some embodiments, the administration of allopregnanolone, e.g., the first or load dose, is initiated within a preselected period of time, wherein said period begins with: the administration of said anesthetic; or the induction of general anesthesia. In some embodiments, said preselected period is not longer than 48, 24, 12, 6, 5, 4, 3, 2, or 1 hour. In some embodiments, said preselected period is not longer than 120, 60, 30, 15, or 5 minutes.

In some embodiments, said second dose is initiated while the subject (e.g., human subject) is under general anesthesia. In some embodiments, the amount of allopregnanolone delivered per hour in said second dose is the same or lower than the amount delivered per hour in said first dose.

In some embodiments, the administration of the first dose of allopregnanolone is initiated within a preselected period of time, wherein said period begins with: the administration of said anesthetic or the induction of general anesthesia. In some embodiments, said preselected period is at least 6, 12, 24, 48 or 60 hours. In some embodiments, said preselected period is not longer than 24, 48, or 60 hours. In some embodiments, said preselected period is between 2 to 120, 2 to 60, 4 to 120, 4 to 60, 4 to 48, 4 to 36, or 4 to 24 hours. In some embodiments, said preselected period is not longer than 48, 24, 12, 6, 5, 4, 3, 2, or 1 hour. In some embodiments, said preselected period is not longer than 120, 60, 30, 15, or 5 minutes.

In some embodiments, said first dose is begun after failure of the subject (e.g., human subject) to respond to prior treatment. In some embodiments, the failure to respond is evidenced by one or more of, a failure to induce an EEG pattern of burst suppression, failure to control seizure, continued seizure activity on EEG recording after 24 hours or more on the first line treatment, or failure to wean from the first line treatment without resuming seizure activity as evidenced by EEG recording. In some embodiments, said prior treatment comprises administration of a first line treatment, e.g., a benzodiazepine (e.g. midazolam). In some embodiments, said prior treatment comprises administration of a second line treatment, e.g., phenytoin, fos-phenytoin, valproate, phenobarbitol, or levetiracetam.

In some embodiments, said first dose is a load, e.g., bolus, dose. In some embodiments, said first dose results in a plasma concentration of 50 to 500 nM, 100 to 400 nM, or 200 to 300 nM. In some embodiments, said first dose results in a plasma concentration of 500 to 1000 nM, 600 to 900 nM, or 700 to 800 nM. In some embodiments, said first dose results in a plasma concentration of 1000 to 1500 nM, 1100 to 1400 nM, or 1200 to 1300 nM. In some embodiments, said first dose results in a plasma concentration of 1500 to 2000 nM, 1600 to 1900 nM, or 1700 to 1800 nM. In some embodiments, said first dose results in a plasma concentration of 2000 to 2500 nM, 2100 to 2400 nM, or 2200 to 2300 nM. In some embodiments, said first dose results in a plasma concentration of 300 to 800 nM, 400 to 700 nM, or 500 to 600 nM. In some embodiments, said first dose results in a plasma concentration of 800 to 1300 nM, 900 to 1200 nM, or 1000 to 1100 nM. In some embodiments, said first dose results in a plasma concentration of 1300 to 1800 nM, 1400 to 1700 nM, or 1500 to 1600 nM. In some embodiments, said first dose results in a plasma concentration of 1800 to 2300 nM, 1900 to 2200 nM, or 2000 to 2100 nM. In some embodiments, said first dose results in a plasma concentration of 2300 to 2600 nM, 2400 to 2500 nM. In some embodiments, said first dose results in a plasma concentration of 300 to 400 nM, 400 to 500 nM, 600 to 700 nM, 800 to 900 nM, 1100 to 1200 nM, 1300 to 1400 nM, 1400 to 1500 nM, 1600 to 1700 nM, 1800 to 1900 nM, 1900 to 2000 nM, 2100 to 2200 nM, 2300 to 2400 nM. In some embodiments, said first dose results in a plasma concentration of 500 to 2500 nM, 500 to 1500 nM, 500 to 1000 nM, 500 to 800, or 500 to 600, nM. In some embodiments, said first dose results in a plasma concentration of 50 to 250 nM, 100 to 200 nM, or 140 to 160 nM. In some embodiments, said first dose results in a plasma concentration of 150+/−30 nM, 150+/−20 nM, 150+/−10 nM, or 150 nM.

In some embodiments, the plasma concentration of said first dose is measured at a preselected time, e.g., at 10, 15, 20, 30, 45, 60 minutes, 2, 3, 4, 5, 6, 8, 10, 12, 24 hours, 2, 3, 4 days after the initiation of said first dose.

In some embodiments, said first dose is administered over a period of time that is not longer than 6, 5, 4, 3, 2, or 1 hour. In some embodiments, said first dose is administered over a period of time that is at least 10, 20, 30, 40, 50, 60, 70, 80, or 90 minutes in duration. In some embodiments, said first dose is administered over a period of time that is 30 to 120 minutes, 45 to 100 minutes, or 50 to 70 minutes, in duration. In some embodiments, said first dose is administered over a period of time that is 60+/−15 minutes, 60+/−10 minutes, 60+/−5 minutes, or 60 minutes, in duration.

In some embodiments, said first dose is administered at a dosage rate of 200-3500 μg/kg/hour. In some embodiments, said first dose is administered at a dosage rate of 200-350 μg/kg/hour, 250-300 μg/kg/hour, 280-290 μg/kg/hour, 286 μg/kg/hour, 287 μg/kg/hour, or 288 μg/kg/hour, e.g., for one hour.

In some embodiments, said second dose is a maintenance dose. In some embodiments, the administration said second dose is initiated within a preselected time period, wherein said time period begins with the administration of said anesthetic. In some embodiments, the administration said second dose is initiated within a preselected time period, wherein said time period begins with the induction of general anesthesia. In some embodiments, the administration said second dose is initiated within a preselected time period, wherein said time period begins with the beginning of the first dose. In some embodiments, the administration said second dose is initiated within a preselected time period, wherein said time period begins with the end of the first dose. In some embodiments, the administration said second dose is initiated within a preselected time period, wherein said time period begins with the achievement of a predetermined level of allopregnanolone, e.g., in the plasma. In some embodiments, said time period begins with the end of the first dose. In some embodiments, said preselected time period begins with beginning or ending of the administration of the first dose and is not longer than 240, 180, 120, 60, 30, 15, or 5 minutes. In some embodiments, said preselected time period begins with beginning or ending of the administration of the first dose and is not longer than 90, 80, 70, or 60 minutes. In some embodiments, the administration of the second dose begins no longer than 90, 80, 70, 60, or 30 minutes after the beginning or end of the administration of the first dose. In some embodiments, the administration of the second dose begins 50 to 70, 55 to 65, or 60 minutes after the beginning or end of the administration of the first dose. In some embodiments, the administration of the second dose begins no more than 60, 50, 40, 30, 20, 10, 5, 4, 3, 2, 1 minute after the end of administration of the first dose. In some embodiments, the administration of the second dose begins at the end of administration of the first dose.

In some embodiments, the administration of the first dose and the initiation of second dose are performed with the same delivery device, e.g., with the same cannula or reservoir.

In some embodiments, said second dose is administered for a period of time that is between 48 and 192 hours, 60 and 144 hours, 60 and 120 hours, 80 and 110 hours, and 90 and 100 hours. In some embodiments, said second dose is administered for 95+/−5 hours. In some embodiments, said second dose is administered for 95 hours.

In some embodiments, said second dose results in a plasma concentration of 50 to 500 nM, 100 to 400 nM, or 200 to 300 nM. In some embodiments, said second dose results in a plasma concentration of 500 to 1000 nM, 600 to 900 nM, or 700 to 800 nM. In some embodiments, said second dose results in a plasma concentration of 1000 to 1500 nM, 1100 to 1400 nM, or 1200 to 1300 nM. In some embodiments, said second dose results in a plasma concentration of 1500 to 2000 nM, 1600 to 1900 nM, or 1700 to 1800 nM. In some embodiments, said second dose results in a plasma concentration of 2000 to 2500 nM, 2100 to 2400 nM, or 2200 to 2300 nM. In some embodiments, said second dose results in a plasma concentration of 300 to 800 nM, 400 to 700 nM, or 500 to 600 nM. In some embodiments, said second dose results in a plasma concentration of 800 to 1300 nM, 900 to 1200 nM, or 1000 to 1100 nM. In some embodiments, said first dose results in a plasma concentration of 1300 to 1800 nM, 1400 to 1700 nM, or 1500 to 1600 nM. In some embodiments, said second dose results in a plasma concentration of 1800 to 2300 nM, 1900 to 2200 nM, or 2000 to 2100 nM. In some embodiments, said second dose results in a plasma concentration of 2300 to 2600 nM, 2400 to 2500 nM. In some embodiments, said second dose results in a plasma concentration of 300 to 400 nM, 400 to 500 nM, 600 to 700 nM, 800 to 900 nM, 1100 to 1200 nM, 1300 to 1400 nM, 1400 to 1500 nM, 1600 to 1700 nM, 1800 to 1900 nM, 1900 to 2000 nM, 2100 to 2200 nM, 2300 to 2400 nM. In some embodiments, said second dose results in a plasma concentration of 500 to 2500 nM, 500 to 1500 nM, 500 to 1000 nM, 500 to 800 nM, or 500 to 600 nM. In some embodiments, said second dose results in a plasma concentration of 50 to 250 nM, 100 to 200 nM, or 140 to 160 nM. In some embodiments, said second dose results in a plasma concentration of 150+/−30 nM, 150+/−20 nM, 150+/−10 nM, or 150 nM.

In some embodiments, the plasma concentration of said second dose is measured at a preselected time, e.g., at 10, 15, 20, 30, 45, 60 minutes, 2, 3, 4, 5, 6, 8, 10, 12, 24 hours, 2, 3, 4 days after the initiation of said second dose.

In some embodiments, said second dose results in a plasma concentration of 150 nM, e.g., as measured at a preselected time, e.g., at 10, 15, 20, 30, 45, 60 minutes, 2, 3, 4, 5, 6, 8, 10, 12, 24 hours, 2, 3, 4 days after the initiation of said second dose.

In some embodiments, said second dose is administered at the same infusion rate, e.g. amount of allopregnanolone/unit time, over the entire second dose. In some embodiments, the infusion rate, e.g. amount of allopregnanolone delivered/unit time varies during the second dose. In some embodiments, said second dose is administered at an infusion rate, e.g. amount of allopregnanolone/unit time of 25-1500 μg/kg/hour. In some embodiments, said second dose is administered at an infusion rate, e.g. amount of allopregnanolone/unit time of 25-150 μg/kg/hour, 50-100 μg/kg/hour, 75-100 μg/kg/hour, 85 μg/kg/hour, 86 μg/kg/hour, or 87 μg/kg/hour.

In some embodiments, said downward taper dose comprises administering a continuously decreasing amount allopregnanolone. In some embodiments, said downward taper dose comprises administering a continuously decreasing amount of allopregnanolone/unit time. In some embodiments, said downward taper dose comprises administering a plurality of step doses, wherein each subsequent step dose is lower than the step dose that precedes it. In some embodiments, said downward taper dose comprises administering a plurality of step doses, wherein each subsequent step dose delivers a lower amount of allopregnanolone/unit time than the step dose that precedes it.

In some embodiments, the method comprises administering a first, second, and third step dose. In some embodiments, said first step dose is 60 to 90% of the second/maintenance dose; said second step dose is 40 to 70% of the second/maintenance dose; and said third step dose is 10 to 40% of the second/maintenance dose. In some embodiments, the amount of allopregnanolone delivered/unit time in said first step dose is 60 to 90% of the amount of allopregnanolone delivered/unit time in said second/maintenance dose; the amount of allopregnanolone delivered/unit time in said second step dose is 40 to 70% of the amount of allopregnanolone delivered/unit time in said second/maintenance dose; and the amount of allopregnanolone delivered/unit time in said third step dose is 10 to 40% of the infusion rate, e.g. amount of allopregnanolone delivered/unit time in said second/maintenance dose. In some embodiments, said first step dose is 70 to 80% of the second/maintenance dose; said second step dose is 40 to 60% of the second/maintenance dose; and said third step dose is 20 to 30% of the second/maintenance dose. In some embodiments, the amount of allopregnanolone delivered/unit time in said first step dose is 70 to 80% of the amount of allopregnanolone delivered/unit time in said second/maintenance dose; the amount of allopregnanolone delivered/unit time in said second step dose is 40 to 60% of the amount of allopregnanolone delivered/unit time in said second/maintenance dose; and the amount of allopregnanolone delivered/unit time in said third step dose is 20 to 30% of the amount of allopregnanolone delivered/unit time in said second/maintenance dose. In some embodiments, said first step dose is 75% of the second/maintenance dose; said second step dose is 50% of the second/maintenance dose; and said third step dose is 25% of the second/maintenance dose. In some embodiments, the amount of allopregnanolone delivered/unit time in said first step dose is 75% of the amount of allopregnanolone delivered/unit time in said second/maintenance dose. In some embodiments, the amount of allopregnanolone delivered/unit time in said second step dose is 50% of the amount of allopregnanolone delivered/unit time in said second/maintenance dose. In some embodiments, the amount of allopregnanolone delivered/unit time in said third step dose is 25% of the amount of allopregnanolone delivered/unit time in said second/maintenance dose.

In some embodiments, after the completion of said third step dose, no allopregnanolone is administered to the subject (e.g., human subject) for at least 10, 20, 30, 40, 50, or 60 days, or until the patient has a subsequent episode of SRSE.

In some embodiments, said first step dose is administered at an amount of allopregnanolone/unit time of 25-1000 μg/kg/hour. In some embodiments, said first step dose is administered at an amount of allopregnanolone/unit time of 25-100 μg/kg/hour, 50-75 μg/kg/hour, 60-70 μg/kg/hour, 63 μg/kg/hour, 64 μg/kg/hour, or 65 μg/kg/hour. In some embodiments, said second step dose is administered at an amount of allopregnanolone/unit time of 10-700 μg/kg/hour. In some embodiments, said second step dose is administered at an amount of allopregnanolone/unit time of 10-70 μg/kg/hour, 25-55 μg/kg/hour, 40-50 μg/kg/hour, 42 μg/kg/hour, 43 μg/kg/hour, or 44 μg/kg/hour. In some embodiments, said third step dose is administered at an amount of allopregnanolone/unit time of 5-500 μg/kg/hour. In some embodiments, said third step dose is administered at an amount of allopregnanolone/unit time of 5-50 μg/kg/hour, 10-35 μg/kg/hour, 15-25 μg/kg/hour, 20 μg/kg/hour, 21 μg/kg/hour, or 22 μg/kg/hour.

In some embodiments, the third/taper dose begins no longer than 90, 80, 70, 60, or 30 minutes after the administration or end of the second dose. In some embodiments, the third/taper dose begins at the end of administration of the second dose.

In some embodiments, the administration of the second dose and the initiation of third/taper dose are performed with the same delivery device, e.g., the same cannula.

In some embodiments, the time between the end of the administration of said first step dose and the initiation of administration of said second step dose is less than 120, 60, 30, 15 or 5 minutes.

In some embodiments, the time between the end of the administration of said second step dose and the initiation of administration of said third step dose is less than 120, 60, 30, 15 or 5 minutes.

In some embodiments, said third dose is administered for a period of time that is between 10 and 100 hours, 12 and 96 hours, 12 and 48 hours, 16 and 32 hours, or 20 and 30 hours.

In some embodiments, said third dose is administered over 24 hours.

In some embodiments, the allopregnanolone is provided in a composition comprising a cyclodextrin, e.g., β-cyclodextrin, e.g., sulfo butyl ether β-cyclodextrin, e.g., CAPTISOL. In some embodiments, the allopregnanolone is provided at a concentration of 0.1 to 10 mg/mL allopregnanolone. In some embodiments, the allopregnanolone is provided at a concentration of 0.1, 0.5, 1, 1.25, 2.5, 3.75, 5, 6.25, 7.5, 8, 9, or 10 mg/mL allopregnanolone. In some embodiments, the allopregnanolone is provided at a concentration of 1.25 mg/mL allopregnanolone. In some embodiments, the allopregnanolone is provided at a concentration of 2.5 mg/mL allopregnanolone. In some embodiments, the allopregnanolone is provided at a concentration of 3.75 mg/mL allopregnanolone. In some embodiments, the allopregnanolone is provided at a concentration of 5 mg/mL allopregnanolone.

In some embodiments, the cyclodextrin is present in the composition at 1-30%, 2-18%, 10-15% by weight of cyclodextrin per volume of composition. In some embodiments, the cyclodextrin is present in the composition at 1, 2.5, 5, 10, 12, 13, 15, 30% by weight of cyclodextrin per volume of composition. In some embodiments, the cyclodextrin is present in the composition at 12% by weight of cyclodextrin per volume of composition.

In some embodiments, the cyclodextrin is present in the composition at 1-30%, 2-18%, 10-15% by weight of cyclodextrin per volume of composition and the allopregnanolone is provided at a concentration of 0.1, 0.5, 1, 1.25, 2.5, 3.75, 5, 6.25, 7.5, 8, 9, or 10 mg/mL allopregnanolone. In some embodiments, the cyclodextrin is present in the composition at 1, 2.5, 5, 10, 12, 13, 15, 30% by weight of cyclodextrin per volume of composition and the allopregnanolone is provided at a concentration of 0.1, 0.5, 1, 1.25, 2.5, 3.75, 5, 6.25, 7.5, 8, 9, or 10 mg/mL allopregnanolone.

In some embodiments, the cyclodextrin is present in the composition at 12% by weight of cyclodextrin per volume of composition and the allopregnanolone is provided at a concentration of 5 mg/mL allopregnanolone. In some embodiments, the cyclodextrin is present in the composition at 12% by weight of cyclodextrin per volume of composition and the allopregnanolone is provided at a concentration of 3.75 mg/mL allopregnanolone. In some embodiments, the cyclodextrin is present in the composition at 12% by weight of cyclodextrin per volume of composition and the allopregnanolone is provided at a concentration of 2.5 mg/mL allopregnanolone. In some embodiments, the cyclodextrin is present in the composition at 12% by weight of cyclodextrin per volume of composition and the allopregnanolone is provided at a concentration of 1.25 mg/mL allopregnanolone.

In some embodiments, the method further comprises, evaluating the subject (e.g., human subject), wherein the evaluating comprises performing c-ECG. In some embodiments, the method comprises, evaluating the subject (e.g., human subject), wherein the evaluating comprises performing EEG. In some embodiments, the method further comprises evaluating the subject (e.g., human subject) for serum chemistry (e.g., one or more of albumin, AST, ALT, bicarbonate, bilirubin, BUN, calcium, chloride, creatine kinase, lipase, creatinine, magnesium, potassium, sodium, total protein, or glucose). In some embodiments, the method further comprises, evaluating the subject (e.g., human subject) for CBC (e.g., one or more of RBC, hemoglobin, hematocrit, MCV, MCH, MCHC, platelet count, WBC with differential including neutrophils, eosinophils, basophils, lymphocytes, or monocytes. In some embodiments, the method further comprises evaluating the subject (e.g., human subject) for serum allopregnanolone, progesterone, and 5α-dihydrotestosterone. In some embodiments, the method comprises comparing an observed value with a reference value.

In some embodiments, said subject (e.g., human subject) is evaluated for a parameter described herein during said weaning period.

In one aspect, the invention features a method of treating a subject (e.g., human subject) having, SE, RSE, or SRSE comprising: administering a first/load, e.g., bolus, dose concurrent with general anesthesia, wherein administration of said first dose: begins 2-120 hours after induction of general anesthesia; lasts for 30-90 minutes; and results in a plasma level of allopregnanolone of 100-2000 nM allopregnanolone; administering a second/maintenance dose, wherein, the administration of said second dose begins not longer than 1-60 minutes after the end of the second dose; lasts for 1-6 days; and results in a plasma level of allopregnanolone of 100-2000 nM allopregnanolone; administering a third downward taper dose, wherein, the administration of said third downward taper dose begins not longer than 1-60 minutes after the end of the third dose; lasts for 10-100 hours; and results in a plasma level of allopregnanolone of 0-1500 nM allopregnanolone; wherein, collectively, the administrations are provided in sufficient amount to treat said subject (e.g., human subject).

In some embodiments, the method comprises administering a first/load, e.g., bolus, dose concurrent with general anesthesia, wherein administration of said first dose: begins 2-120 hours after induction of general anesthesia; lasts for 60+/−15 minutes; administering a second/maintenance dose, wherein, the administration of said second dose begins not longer than 30 minutes after the end of the second dose; lasts for 70 to 110 hours; administering a third downward taper dose, wherein, the administration of said third downward taper dose begins not longer than 1-60 minutes after the end of the third dose; lasts for 10-30 hours.

In some embodiments, administering a first/load, e.g., bolus, dose concurrent with general anesthesia, wherein administration of said first dose: begins 2-120 hours after induction of general anesthesia; lasts for 60+/−15 minutes; administering a second/maintenance dose, wherein, the administration of said second dose begins not longer than 30 minutes after the end of the second dose; lasts for 70 to 110 hours; and administering a third downward taper dose, wherein, the administration of said third downward taper dose begins not longer than 1-60 minutes after the end of the third dose; lasts for 24+/−2 hours and said third downward taper dose comprises a first, second, and third step dose.

In some embodiments, administering a first/load, e.g., bolus, dose concurrent with general anesthesia, wherein administration of said first dose lasts for 60+/−15 minutes; administering a second/maintenance dose, wherein, the administration of said second dose begins not longer than 30 minutes after the end of the second dose; lasts for 85 to 105 hours; administering a third downward taper dose, wherein, the administration of said third downward taper dose begins not longer than 1-60 minutes after the end of the third dose; lasts for 10-30 hours and: the amount of allopregnanolone delivered/unit time in said first step dose is 70 to 80% of the amount of allopregnanolone delivered/unit time in said second/maintenance dose; the amount of allopregnanolone delivered/unit time in said second step dose is 40 to 60% of the amount of allopregnanolone delivered/unit time in said second/maintenance dose; and the amount of allopregnanolone delivered/unit time in said third step dose is 20 to 30% of the amount of allopregnanolone delivered/unit time in said second/maintenance dose.

In some embodiments, administering a first/load, e.g., bolus, dose concurrent with general anesthesia, wherein administration of said first dose lasts for 60+/−5 minutes; administering a second/maintenance dose, wherein, the administration of said second dose begins not longer than 30 minutes after the end of the second dose; lasts for 96+/−4 hours; administering a third downward taper dose, wherein, the administration of said third downward taper dose begins not longer than 1-60 minutes after the end of the third dose; lasts for 24+/−2 hours and: the amount of allopregnanolone delivered/unit time in said first step dose is 75% of the amount of allopregnanolone delivered/unit time in said second/maintenance dose; the amount of allopregnanolone delivered/unit time in said second step dose is 50% of the amount of allopregnanolone delivered/unit time in said second/maintenance dose; and the amount of allopregnanolone delivered/unit time in said third step dose is 25% of the amount of allopregnanolone delivered/unit time in said second/maintenance dose.

In some embodiments, the method further comprises administering an amount of a composition selected from benzodiazepines (e.g., midazolam), propofol, barbiturates, and ketamine sufficient to place said subject (e.g., human subject) under general anesthesia;

In one aspect, the invention features a kit comprising one or more of: a preparation of allopregnanolone, e.g., a plurality of preparations of allopregnanolone at a concentrations suitable for use at the first, second, and third doses; and instructions for use for treating a subject (e.g., human subject) having a seizure-related disorder, e.g., status epilepticus (SE), e.g., super-refractory status epilepticus (SRSE), wherein said seizure-related disorder is preceded by a condition related to a structural modification in the brain of said subject.

In one aspect, the invention features a kit comprising one or more of: a preparation of allopregnanolone, and instructions for use for treating a subject having a seizure-related disorder, wherein said said seizure-related disorder is preceded by a condition selected from the group consisting of a brain aneurysm with associated hemorrhage, an intraparenchymal hemorrhage, a brain arteriovenous malformation with associated hemorrhage, an ischemic stroke, a focal cortical dysplasias or malformation of cortical development, an intraparenchymal brain tumor, post-traumatic porenenchephaly or gliosis, a cerebral abscess, a central nervous system infection, encephalitis, multiple sclerosis, and a demyelinating lesion, the method comprising: administering to said subject, an effective amount of allopregnanolone, thereby treating said subject.

In some embodiments, the above-described kits further comprise a suitable diluent (e.g., water, saline, cyclodextrin, e.g., β-cyclodextrin, e.g., sulfo butyl ether β-cyclodextrin, e.g., CAPTISOL).

In some embodiments, the allopregnanolone is provided at a concentration of 0.1-10 mg/mL allopregnanolone. In some embodiments, the allopregnanolone is provided at a concentration of 0.5-7.5 mg/mL allopregnanolone. In some embodiments, the allopregnanolone is provided at a concentration of 1-6 mg/mL allopregnanolone. In some embodiments, the allopregnanolone is provided at a concentration of 5 mg/mL allopregnanolone. In some embodiments, the allopregnanolone is provided at a concentration of 3.75 mg/mL allopregnanolone. In some embodiments, the allopregnanolone is provided at a concentration of 2.5 allopregnanolone. In some embodiments, the allopregnanolone is provided at a concentration of 1.25 mg/mL allopregnanolone.

In one aspect, the invention features a method of treating a subject having seizure, epilepsy or status epilepticus by administering in combination to the subject allopregnanolone and a benzodiazepine or anesthetic/sedative. In some embodiments, the method further comprises administering at least one of the allopregnanolone and benzodiazepine or anesthetic/sedative parenterally (e.g., intranasally, buccally, intravenously or intramuscularly, for example, intravenously or intramuscularly). In some embodiments, both the allopregnanolone and benzodiazepine or anesthetic/sedative are administered parenterally.

In some embodiments, the allopregnanolone and benzodiazepine or anesthetic/sedative co-administered (e.g., administered simultaneously, administered concurrently). In some embodiments, the allopregnanolone and benzodiazepine or anesthetic/sedative are administered sequentially. In some embodiments, allopregnanolone and benzodiazepine or anesthetic/sedative are administered in a single dosage form.

When the agents described herein (e.g., the allopregnanolone and a benzodiazepine or anesthetic/sedative) are administered in combination, both of the agents should be present at dosage levels of between about 1 to 100%, and more preferably between about 5 to 95% of the dosage normally administered in the absence of the combination regimen. The agents may be administered separately, as part of a multiple dose regimen. Alternatively, the agents may be part of a single dosage form, mixed together with the compounds of this invention in a single composition.

In some embodiments, the allopregnanolone is a progestin derivative, e.g., allopregnanolone. In an embodiment, the allopregnanolone is allopregnanolone.

In some embodiments, the allopregnanolone is formulated for parenteral administration (e.g., intranasally, buccally, intravenously or intramuscularly, for example, intravenously or intramuscularly).

In some embodiments, the allopregnanolone is administered in a composition comprising a cyclodextrin, e.g., a β-cyclodextrin, e.g., a sulfo butyl ether β-cyclodextrin, e.g., CAPTISOL®, complex.

In some embodiments, the cyclodextrin is a β-cyclodextrin. In an embodiment, the cyclodextrin is a sulfo butyl ether β-cyclodextrin. In an embodiment, the cyclodextrin is CAPTISOL®. In some embodiments, the cyclodextrin is a β-cyclodextrin disclosed in U.S. Pat. Nos. 5,874,418; 6,046,177; or 7,635,733, which are herein incorporated by reference. In some embodiments, the allopregnanolone is a progestin derivative, and the cyclodextrin is a β-cyclodextrin. In some embodiments, the allopregnanolone is a progestin derivative, and the cyclodextrin is a sulfo butyl ether β-cyclodextrin. In an embodiment, the allopregnanolone is allopregnanolone and the cyclodextrin is CAPTISOL®.

In some embodiments, the allopregnanolone and cyclodextrin, e.g., a β-cyclodextrin, e.g., a sulfo butyl ether β-cyclodextrin, e.g., CAPTISOL®, complex is formulated for parenteral administration. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition. In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration between 0.25-30 mg/mL, 0.5-30 mg/mL; 1-30 mg/mL; 5-30 mg/mL, 10-30 mg/mL; 15-30 mg/mL, 0.25-20 mg/mL; 0.5-20 mg/mL; 1-20 mg/mL, 0.5-20 mg/mL; 1-20 mg/mL, 5-20 mg/mL, 10-20 mg/mL, 0.25-15 mg/mL, 0.5-15 mg/mL; 0.5-10 mg/mL; 0.5-7 mg/mL; 1-15 mg/mL, 1-10 mg/mL; 1-7 mg/mL; 1-5 mg/mL; 5-15 mg/mL; 5-10 mg/mL; 10-15 mg/mL; 1-10 mg/mL; 2-8 mg/mL; 2-7 mg/mL; 3-5 mg/mL; 5-15 mg/mL; 3-7 mg/mL; 4-6 mg/mL; 7-12 mg/mL; 7-10 mg/mL; 8-9 mg/mL; 3-5 mg/mL; or 3-4 mg/mL. In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 0.25 mg/mL, 0.5 mg/mL; 1.0 mg/mL; 1.5 mg/mL; 2.0 mg/mL; 2.5 mg/mL; 3.0 mg/mL; 3.5 mg/mL; 4.0 mg/mL; 4.5 mg/mL; 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10 mg/mL, 15 mg/mL, 20 mg/mL, 25 mg/mL, or 30 mg/mL. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 1.5 mg/mL. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 2.5 mg/mL. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 3.5 mg/mL. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 5 mg/mL. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 6 mg/mL. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 15 mg/mL.

In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of between 0.1-50 μM; 0.1-40 μM; 0.1-30 μM; 0.1-20 μM; 0.1-15 μM; 0.5-50 μM; 0.5-40 μM; 0.5-30 μM; 0.5-20 μM; 0.5-15 μM; 1-50 μM; 1-40 μM; 1-30 μM; 1-20 μM; 1-15 μM; 2-50 μM; 2-40 μM; 2-30 μM; 2-20 μM; 2-15 μM; 0.5-15 μM; 1-15 μM; 2-15 μM; 3-15 μM; 1-20 μM. In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 0.1 μM; 0.5-1 μM; 2 μM; 4 μM; 5 μM; 7 μM; 10 μM; 15 μM; 20 μM; 25 μM; 40 μM; 50 μM. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 1 μM. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 2 μM. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 5 μM.

In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising the cyclodextrin, e.g., a β-cyclodextrin, e.g., a sulfo butyl ether β-cyclodextrin, e.g., CAPTISOL®, at a concentration between 25-400 mg/mL; 25-300 mg/mL; 25-200 mg/mL; 25-100 mg/mL; 25-50 mg/mL; 50-400 mg/mL; 50-300 mg/mL; 60-400 mg/mL; 60-300 mg/mL; 150-400 mg/mL; 150-300 mg/mL; 200-300 mg/mL; 200-400 mg/mL; 30-100 mg/mL; 30-300 mg/mL; 30-400 mg/mL; 45-75 mg/mL; 50-70 mg/mL; 55-65 mg/mL; or 50-60 mg/mL. In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising the cyclodextrin, e.g., a β-cyclodextrin, e.g., a sulfo butyl ether β-cyclodextrin, e.g., CAPTISOL®, at a concentration of 25 mg/mL; 30 mg/mL; 35 mg/mL; 40 mg/mL; 45 mg/mL; 50 mg/mL; 55 mg/mL; 60 mg/mL; 65 mg/mL; 70 mg/mL; 75 mg/mL; 80 mg/mL; 85 mg/mL; 90 mg/mL, 95 mg/mL; 100 mg/mL; 150 mg/mL; 200 mg/mL; 250 mg/mL; 300 mg/mL; 350 mg/mL; or 400 mg/mL. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the cyclodextrin, e.g., a β-cyclodextrin, e.g., a sulfo butyl ether β-cyclodextrin, e.g., CAPTISOL®, at a concentration of 60 mg/mL.

In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising between 2.5-40%, 2.5-30%, 2.5-20%, 2.5-15%, 2.5-10%, 5-40%, 5-30%, 5-20%, 5-15%, 5-10%, 6-40%, 6-30%, 6-20%, 6-10%, 6-20%, 6-30%, 10-40%, 10-30%, 10-20%, 20-40%, 20-30%, 25-40%, 25-30%, 3-10%, 3-15%, 4.5-7.5%, 4-13%, 5-7%, 5-13%, 5.5-6.5%, 7-13% by weight of the cyclodextrin, e.g., CAPTISOL® per weight of solution.

In some embodiments, the allopregnanolone and cyclodextrin, e.g., a β-cyclodextrin, e.g., a sulfo butyl ether β-cyclodextrin, e.g., CAPTISOL®, complex is formulated as an aqueous composition comprising 2.5%, 3%, 4%, 4.5%, 5%, 5.5%, 6%, 6.5%, 7%, 7.5%, 8%, 8.5%, 9%, 9.5%, 10%, 12%, 15%, 20%, 25%, 30%, 35% or 40% by weight of the cyclodextrin, e.g., CAPTISOL® per weight of solution. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising 6% by weight of the cyclodextrin, e.g., CAPTISOL® per weight of solution. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising 12% by weight of the cyclodextrin, e.g., CAPTISOL® per weight of solution. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising 15% by weight of the cyclodextrin, e.g., CAPTISOL® per weight of solution. In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising 30% by weight of the cyclodextrin, e.g., CAPTISOL® per weight of solution.

In an embodiment, the allopregnanolone and cyclodextrin, e.g., a β-cyclodextrin, e.g., a sulfo butyl ether β-cyclodextrin, e.g., CAPTISOL®, complex is formulated as an aqueous composition comprising the allopregnanolone at a concentration of 1.5 mg/mL, and the cyclodextrin, e.g., a β-cyclodextrin, e.g., a sulfo butyl ether β-cyclodextrin, e.g., CAPTISOL®, at a concentration of 6% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 10 mg/mL, and the cyclodextrin, e.g., a β-cyclodextrin, e.g., a sulfo butyl ether β-cyclodextrin, e.g., CAPTISOL®, at a concentration of 6% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 15 mg/mL, and the cyclodextrin, e.g., a β-cyclodextrin, e.g., a sulfo butyl ether β-cyclodextrin, e.g., CAPTISOL®, at a concentration of 6% by weight of the cyclodextrin, e.g., CAPTISOL® per weight of solution.

In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 1.25 mg/mL, and the cyclodextrin, e.g., a β-cyclodextrin, e.g., a sulfo butyl ether β-cyclodextrin, e.g., CAPTISOL®, at a concentration of 12% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 1.5 mg/mL, and the cyclodextrin, e.g., a β-cyclodextrin, e.g., a sulfo butyl ether β-cyclodextrin, e.g., CAPTISOL®, at a concentration of 12% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 2.5 mg/mL, and the cyclodextrin, e.g., a β-cyclodextrin, e.g., a sulfo butyl ether β-cyclodextrin, e.g., CAPTISOL®, at a concentration of 12% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 3.75 mg/mL, and the cyclodextrin, e.g., a β-cyclodextrin, e.g., a sulfo butyl ether β-cyclodextrin, e.g., CAPTISOL®, at a concentration of 12% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 5 mg/mL, and the cyclodextrin, e.g., a β-cyclodextrin, e.g., a sulfo butyl ether β-cyclodextrin, e.g., CAPTISOL®, at a concentration of 12% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 10 mg/mL, and the cyclodextrin, e.g., a β-cyclodextrin, e.g., a sulfo butyl ether β-cyclodextrin, e.g., CAPTISOL®, at a concentration of 12% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 15 mg/mL, and the cyclodextrin, e.g., a β-cyclodextrin, e.g., a sulfo butyl ether β-cyclodextrin, e.g., CAPTISOL®, at a concentration of 12% by weight of the cyclodextrin, e.g., CAPTISOL® per weight of solution.

In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 1.5 mg/mL, and the cyclodextrin, e.g., a β-cyclodextrin, e.g., a sulfo butyl ether β-cyclodextrin, e.g., CAPTISOL®, at a concentration of 15% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 10 mg/mL, and the cyclodextrin, e.g., a β-cyclodextrin, e.g., a sulfo butyl ether β-cyclodextrin, e.g., CAPTISOL®, at a concentration of 15% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 15 mg/mL, and the cyclodextrin, e.g., a β-cyclodextrin, e.g., a sulfo butyl ether β-cyclodextrin, e.g., CAPTISOL®, at a concentration of 15% by weight of the cyclodextrin, e.g., CAPTISOL® per weight of solution.

In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 1.5 mg/mL, and the cyclodextrin, e.g., a β-cyclodextrin, e.g., a sulfo butyl ether β-cyclodextrin, e.g., CAPTISOL®, at a concentration of 30% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 10 mg/mL, and the cyclodextrin, e.g., a β-cyclodextrin, e.g., a sulfo butyl ether β-cyclodextrin, e.g., CAPTISOL®, at a concentration of 30% by weight of the cyclodextrin, e.g., CAPTISOL® per weight of solution. In an embodiment, the allopregnanoloneand cyclodextrin, e.g., a β-cyclodextrin, e.g., a sulfo butyl ether β-cyclodextrin, e.g., CAPTISOL®, complex is formulated as an aqueous composition comprising the allopregnanoloneat a concentration of 15 mg/mL, and the cyclodextrin, e.g., a β-cyclodextrin, e.g., a sulfo butyl ether β-cyclodextrin, e.g., CAPTISOL®, at a concentration of 30% by weight of the cyclodextrin, e.g., CAPTISOL® per weight of solution.

In some embodiments, the allopregnanolone and CAPTISOL® complex is formulated as an aqueous composition with a pH between 3-10, 4-9, 4-8, 4-7, 4-6, 4-5, 5-9, 5-8, 5-7, 5-6, 4.5-7.5, or 5.5-7.5. In some embodiments, the allopregnanolone and CAPTISOL® complex is formulated as an aqueous composition with a pH about 3, 3.5, 4, 4.5, 5, 5.5, 6, 6.5, 7, 7.5, 8, 8.5, or 9. In an embodiment, the allopregnanolone and CAPTISOL® complex is formulated as an aqueous composition with a pH about 6.

In some embodiments, the allopregnanolone and CAPTISOL® complex is formulated as an aqueous composition and is administered intravenously. In some embodiments, the allopregnanolone and CAPTISOL® complex is formulated as an aqueous composition and is administered intramuscularly.

In some embodiments, the allopregnanolone and CAPTISOL® complex is formulated as an aqueous composition and is administered for 1, 2, 3, 4, 5, 6, 7, 8, 9, or 10 consecutive days. In some embodiments, the allopregnanolone and CAPTISOL® complex is formulated as an aqueous composition and is administered between 1-10, 1-5, 5-10, 1-6, 2-6, 3-6, 4-5, or 1-9 consecutive days. In an embodiment, the allopregnanolone and CAPTISOL® complex is formulated as an aqueous composition and is administered for 5 consecutive days. In some embodiments, the duration of administration is 1, 2, 3, 4, 5, 6, 7, 8, 9, or 10 days. In some embodiments, the duration of administration is 3-7, 4-6, 4-5, or 5-6 days. In some embodiments, the duration of administration is 5 days.

In some embodiments, the allopregnanolone and CAPTISOL® complex is formulated as an aqueous composition and is administered at the same dose for 1, 2, 3, 4, 5, 6, 7, 8, 9, or 10 consecutive days. In some embodiments, the allopregnanolone and CAPTISOL® complex is formulated as an aqueous composition and is administered at a load, e.g., bolus, dose for 1, 2, 3, 4, 5, 6, 7, 8, 9, or 10 consecutive days and then administered at a maintenance, e.g., infusion, dose for 1, 2, 3, 4, 5, 6, 7, 8, 9, or 10 consecutive days. In an embodiment, the allopregnanolone and CAPTISOL® complex is formulated as an aqueous composition and is administered at a load, e.g., bolus, dose of 0.25 mg/mL, 0.5 mg/mL; 1.0 mg/mL; 1.5 mg/mL; 2.0 mg/mL; 2.5 mg/mL; 3.0 mg/mL; 3.5 mg/mL; 4.0 mg/mL; 4.5 mg/mL; 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10 mg/mL, 15 mg/mL, 20 mg/mL, 25 mg/mL, or 30 mg/mL allopregnanolone for 1 day and then administered at a maintenance, e.g., infusion, dose for 1, 2, 3, 4, 5, or 6 consecutive days of 0.25 mg/mL, 0.5 mg/mL; 1.0 mg/mL; 1.5 mg/mL; 2.0 mg/mL; 2.5 mg/mL; 3.0 mg/mL; 3.5 mg/mL; 4.0 mg/mL; 4.5 mg/mL; 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10 mg/mL, 15 mg/mL, 20 mg/mL, 25 mg/mL, or 30 mg/mL allopregnanolone. In some embodiments, a maintenance, e.g., infusion, dose described herein, is lower than a load, e.g., bolus, dose described herein. In some embodiments, a maintenance, e.g., infusion, dose described herein, is the same as a load, e.g., bolus, dose described herein. In some embodiments, the maintenance, e.g., infusion, dose is less than 0.25 mg/mL, 0.5 mg/mL; 1.0 mg/mL; 1.5 mg/mL; 2.0 mg/mL; 2.5 mg/mL; 3.0 mg/mL; 3.5 mg/mL; 4.0 mg/mL; 4.5 mg/mL; 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10 mg/mL, 15 mg/mL, 20 mg/mL, 25 mg/mL, or 30 mg/mL.

In some embodiments, the allopregnanolone and CAPTISOL® complex is formulated as an aqueous composition and is administered at a load, e.g., bolus, dose for 1, 2, 3, 4, 5, 6, 7, 8, 9, or 10 consecutive days and then administered at a maintenance, e.g., infusion, dose for 1, 2, 3, 4, 5, 6, 7, 8, 9, or 10 consecutive days and then administered at a taper dose for 1, 2, 3, 4, 5, 6, 7, 8, 9, or 10 consecutive days. In some embodiments, the allopregnanolone and CAPTISOL® complex is formulated as an aqueous composition and is administered at a load, e.g., bolus, dose for 1, 2, 3, 4, 5, 6, 7, 8, 9, or 10 consecutive days and then administered at a maintenance, e.g., infusion, dose for 1, 2, 3, 4, 5, 6, 7, 8, 9, or 10 consecutive days and then administered at a first step dose for 1, 2, 3, 4, 5, 6, 7, 8, 9, or 10 hours.

In some embodiments, the allopregnanolone and CAPTISOL® complex is formulated as an aqueous composition and is administered at a load, e.g., bolus, dose for 1, 2, 3, 4, 5, 6, 7, 8, 9, or 10 consecutive days and then administered at a maintenance, e.g., infusion, dose for 1, 2, 3, 4, 5, 6, 7, 8, 9, or 10 consecutive days and then administered at a first step dose for 1, 2, 3, 4, 5, 6, 7, 8, 9, or 10 hours and then administered at a second step dose for 1, 2, 3, 4, 5, 6, 7, 8, 9, or 10 hours. In some embodiments, the allopregnanolone and CAPTISOL® complex is formulated as an aqueous composition and is administered at a load, e.g., bolus, dose for 1, 2, 3, 4, 5, 6, 7, 8, 9, or 10 consecutive days and then administered at a maintenance, e.g., infusion, dose for 1, 2, 3, 4, 5, 6, 7, 8, 9, or 10 consecutive days and then administered at a first step dose for 1, 2, 3, 4, 5, 6, 7, 8, 9, or 10 hours and then administered at a second step dose for 1, 2, 3, 4, 5, 6, 7, 8, 9, or 10 hours and then administered at a third step dose for 1, 2, 3, 4, 5, 6, 7, 8, 9, or 10 hours.

In some embodiments the first, second, or third step dose is less than the maintenance, e.g., infusion, dose. In some embodiments, the second taper or third step dose is less than the first step dose. In some embodiments, the third step dose is less than the second step dose. In some embodiments, the first step dose is 95%, 90%, 85%, 80%, 75%, 70%, 65%, 60%, 55%, 50%, 45%, 40%, 35%, 30%, 25%, 20%, 15%, 10%, or 5% of the maintenance, e.g., infusion, dose. In some embodiments, the first step dose is between 95-50%, 75-50%, 85-50%, 90-50%, 80-50%, or 75-100% of the maintenance, e.g., infusion, dose. In an embodiment, the first step dose is 75% of the maintenance, e.g., infusion, dose.

In some embodiments, the second step dose is 95%, 90%, 85%, 80%, 75%, 70%, 65%, 60%, 55%, 50%, 45%, 40%, 35%, 30%, 25%, 20%, 15%, 10%, or 5% of the maintenance, e.g., infusion, dose. In some embodiments, the second step dose is between 95-30%, 75-30%, 85-30%, 60-30%, 70-30%, 50-30%, or 50-40% of the maintenance, e.g., infusion, dose. In an embodiment, the second step dose is 50% of the maintenance, e.g., infusion, dose.

In some embodiments, the third step dose is 95%, 90%, 85%, 80%, 75%, 70%, 65%, 60%, 55%, 50%, 45%, 40%, 35%, 30%, 25%, 20%, 15%, 10%, or 5% of the maintenance, e.g., infusion, dose. In some embodiments, the third step dose is between 50-5%, 40-5%, 30-5%, 25-5%, 25-10%, 25-20%, or 25-40% of the maintenance, e.g., infusion, dose. In an embodiment, the second step dose is 50% of the maintenance, e.g., infusion, dose. In an embodiment, the third step dose is 25% of the maintenance, e.g., infusion, dose.

In an embodiment, a composition comprising allopregnanoloneand cyclodextrin, e.g., a β-cyclodextrin, e.g., a sulfo butyl ether β-cyclodextrin, e.g., CAPTISOL®, complex, comprises less than 100 ppm of a phosphate, and the cyclodextrin, e.g., a β-cyclodextrin, e.g., a sulfo butyl ether β-cyclodextrin, e.g., CAPTISOL®, has an absorption of less than 0.2 A.U. due to a drug-degrading agent, as determined by UV/vis spectrophotometry at a wavelength of 245 nm to 270 nm for an aqueous solution comprising 300 mg of the cyclodextrin, e.g., a β-cyclodextrin, e.g., a sulfo butyl ether β-cyclodextrin, e.g., CAPTISOL®, per mL of solution in a cell having a 1 cm path length.

In some embodiments, the cyclodextrin, e.g., a β-cyclodextrin, e.g., a sulfo butyl ether β-cyclodextrin, e.g., CAPTISOL®, has an absorption of less than 0.2 A.U. due to a color forming agent, as determined by UV/vis spectrophotometry at a wavelength of 320 nm to 350 nm for an aqueous solution comprising 500 mg of the cyclodextrin, e.g., a β-cyclodextrin, e.g., a sulfo butyl ether β-cyclodextrin, e.g., CAPTISOL®, per mL of solution in a cell having a 1 cm path length.

In some embodiments, the cyclodextrin, e.g., a β-cyclodextrin, e.g., a sulfo butyl ether β-cyclodextrin, e.g., CAPTISOL®, further comprises: less than 20 ppm of a sulfoalkylating agent; less than 0.5% wt. of an underivatized cyclodextrin; less than 1% wt. of an alkali metal halide salt; and less than 0.25% wt. of a hydrolyzed sulfoalkylating agent.

In some embodiments, the cyclodextrin, e.g., a β-cyclodextrin, e.g., a sulfo butyl ether β-cyclodextrin, e.g., CAPTISOL®, has an absorption of less than 0.2 A.U. due to a drug-degrading agent, as determined by UV/vis spectrophotometry at a wavelength of 245 nm to 270 nm for an aqueous solution comprising 500 mg of the cyclodextrin, e.g., a β-cyclodextrin, e.g., a sulfo butyl ether β-cyclodextrin, e.g., CAPTISOL®, per mL of solution in a cell having a 1 cm path length.

In some embodiments, the cyclodextrin, e.g., a β-cyclodextrin, e.g., a sulfo butyl ether β-cyclodextrin, e.g., CAPTISOL®, further comprises: less than 50 ppm of a phosphate; less than 10 ppm of a sulfoalkylating agent; less than 0.2% wt. of an underivatized cyclodextrin; less than 0.5% wt. of an alkali metal halide salt; and less than 0.1% wt. of a hydrolyzed sulfoalkylating agent; and wherein the cyclodextrin, e.g., a β-cyclodextrin, e.g., a sulfo butyl ether β-cyclodextrin, e.g., CAPTISOL®, has an absorption of less than 0.2 A.U. due to the color-forming agent, as determined by U/vis spectrophotometry at a wavelength of 320 nm to 350 nm for an aqueous solution comprising 500 mg of the cyclodextrin, e.g., a β-cyclodextrin, e.g., a sulfo butyl ether β-cyclodextrin, e.g., CAPTISOL®, per mL of solution in a cell having a 1 cm path length.

In some embodiments, the cyclodextrin, e.g., a β-cyclodextrin, e.g., a sulfo butyl ether β-cyclodextrin, e.g., CAPTISOL®, further comprises: less than 10 ppm of a phosphate; less than 2 ppm of a sulfoalkylating agent; less than 0.1% wt. of an underivatized cyclodextrin; less than 0.2% wt. of an alkali metal halide salt; and less than 0.08% wt. of a hydrolyzed sulfoalkylating agent; and wherein the cyclodextrin, e.g., a β-cyclodextrin, e.g., a sulfo butyl ether β-cyclodextrin, e.g., CAPTISOL®, has an absorption of less than 0.1 A.U. due to the color-forming agent, as determined by UV/vis spectrophotometry at a wavelength of 320 nm to 350 nm for an aqueous solution comprising 500 mg of the cyclodextrin, e.g., a β-cyclodextrin, e.g., a sulfo butyl ether β-cyclodextrin, e.g., CAPTISOL®, per mL of solution in a cell having a 1 cm path length.

In some embodiments, the cyclodextrin, e.g., a β-cyclodextrin, e.g., a sulfo butyl ether β-cyclodextrin, e.g., CAPTISOL®, further comprises: less than 5 ppm of a phosphate; less than 0.1% wt. of an alkali metal halide salt; and less than 0.05% wt. of a hydrolyzed sulfoalkylating agent.

In some embodiments, the allopregnanolone and CAPTISOL® complex is formulated as an aqueous composition and is administered within 48 hours, 24 hours, 10 hours, 8 hours, 5 hours, 3 hours, 1 hour, or 0.5 hour after a seizure, e.g., a status epileptic seizure, e.g., a refractory status epileptic seizure has started. In some embodiments, the allopregnanolone and CAPTISOL® complex is formulated as an aqueous composition and is administered within 60 minutes, 45 minutes, 30 minutes, 15 minutes, 10 minutes, or 5 minutes after a seizure, e.g., a status epileptic seizure, e.g., a refractory status epileptic seizure has started. In some embodiments, the allopregnanolone and CAPTISOL® complex is formulated as an aqueous composition and is administered after a seizure, e.g., a status epileptic seizure, e.g., a refractory status epileptic seizure has lasted 5 minutes, 10 minutes, 15 minutes, 20 minutes, 30 minutes or 60 minutes.

In some embodiments, the allopregnanolone and CAPTISOL® complex is formulated as an aqueous composition and is administered prior to the onset of a seizure, e.g., a status epileptic seizure, e.g., a refractory status epileptic seizure.

In some embodiments, the benzodiazepine is clonazepam, lorazepam, midazolam, or diazepam.

In some embodiments, the benzodiazepine is formulated for oral delivery. In some embodiments, the benzodiazepine is formulated for parenteral delivery (e.g., intranasally, buccally, intravenously or intramuscularly, for example, intravenously or intramuscularly).

In some embodiments, the anesthetic/sedative is propofol or a barbiturate, e.g., pentobarbital.

In some embodiments, both the allopregnanolone and the benzodiazepine or anesthetic/sedative are formulated for parenteral delivery (e.g., intranasally, buccally, intravenously or intramuscularly, for example, intravenously or intramuscularly).

In some embodiments, the allopregnanolone and benzodiazepine or anesthetic/sedative, when administered in combination, are administered in an amount sufficient to achieve burst suppression (e.g., a predetermined burst suppression pattern, e.g., inter-burst intervals of between 2-30 seconds; as measured by a method of neurophysiological monitoring, e.g., EEG, CFM). In some embodiments, the allopregnanolone and benzodiazepine or anesthetic/sedative, when administered in combination, is administered at a dose sufficient to achieve a predetermined burst suppression pattern, e.g., inter-burst intervals of between 2-30 seconds, 5-30 seconds, 10-30 seconds, 15-30 seconds, 1-30 seconds, 0-30 seconds, 2-20 seconds, 2-10 seconds, 5-20 seconds, 10-20 seconds, 15-25 seconds, 5-15 seconds or 5-10 seconds; as measured by a method of neurophysiological monitoring, e.g., EEG, CFM.

### BRIEF DESCRIPTION OF THE DRAWINGS

FIG. 1 depicts exemplary clinical data showing the amount of treatment responders with SAGE-547 injection relative to placebo where the SRSE is preceded by a structural modification in the brain.

FIG. 2 depicts exemplary clinical data showing the amount of treatment responders with SAGE-547 injection relative to placebo where the SRSE is correlated to exemplary etiologies.

### DETAILED DESCRIPTION OF THE INVENTION

As used herein, “administered in combination” or a combined administration of two agents (e.g., allopregnanlone and a benzodiazepine or anesthetic/sedative) means that two or more agents are administered to a subject at the same time or within an interval such that there is overlap of an effect of each agent on the patient. Preferably they are administered within 15, 10, 5, or 1 minute of one another. Preferably the administrations of the agents are spaced sufficiently close together such that a combinatorial effect is achieved. The agents can be administered simultaneously, for example in a combined unit dose (providing simultaneous delivery of both agents). Alternatively, the agents can be administered at a specified time interval, for example, an interval of minutes, hours, days or weeks. Generally, the agents are concurrently bioavailable, e.g., detectable, in the subject. In an embodiment, the agents (e.g., allopregnanolone and a benzodiazepine or anesthetic/sedative) are administered essentially simultaneously, for example two unit dosages administered at the same time, or a combined unit dosage of the two agents. In another embodiment, the agents are delivered in separate unit dosages. The agents can be administered in any order, or as one or more preparations that includes two or more agents. In an embodiment, at least one administration of one of the agents, e.g., the first agent, is made within minutes, one, two, three, or four hours, or even within one or two days of the other agent, e.g., the second agent. In some cases, combinations can achieve synergistic results, e.g., greater than additive results, e.g., at least 20, 50, 70, or 100% greater than additive.

As used herein, “allopregnanolone” refers to the compound 3α-hydroxy-5α-pregnan-20-one.

As used herein, “SAGE-547 injection,” “SAGE-547,” or “Brexanolone” is a solution of 5 mg/mL allopregnanolone in Sterile Water for Injection (SWFI), USP and 250 mg/mL betadex sulfobutyl-ether sodium, NF. It is further diluted with Sterile Water for Injection, USP (SWFI) in IV bags to achieve an allopregnanolone concentration of approximately 1.67 mg/mL in an approximately isotonic solution and will be administered intravenously.

As used herein, “concurrent” administration of a treatment modality with a selected state, e.g., being under general anesthesia, or while a second treatment modality is administered, or is present at a preselected level, e.g., a therapeutic level, means that administration of the treatment modality overlaps or occurs at the same time as, e.g., administration of a second treatment modality.

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

As used herein, “general anesthesia” or “GA” is a state produced when a subject receives medications for e.g., amnesia, analgesia, muscle paralysis, and sedation. For example, GA is a treatment that induces deep sleep typically used so that subjects will not feel pain during surgery. An anesthetized patient can be thought of as being in a reversible and controlled state of unconsciousness. GA agents can be administered intravenously or inhaled.

As used herein, the term “subject” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc.

### Anesthetics and Sedatives

An anesthetic (e.g., general anesthetic) agent or sedative is a drug that can bring about, induce, and maintain a reversible loss of consciousness. A sedative is a substance that induces sedation by reducing irritability or excitement in a subject. Intravenous injections of anesthetics are generally preferred to inhalation, intramuscular or subcutaneous injections because they are faster, generally less painful and more reliable. Exemplary anesthetics include propofol, etomidate, barbiturates (e.g., pentobarbital, methohexital, thiopentone/thiopental), benzodiazepines (e.g., as described herein, e.g., midazolam), and ketamine.

In some embodiments, a subject that has been administered an anesthetic agent or anesthetic is under general anesthesia.

### Benzodiazepines

A benzodiazepine is a compound having a core chemical structure of a fusion of a benzene ring and a diazepine ring. The first benzodiazepine, chlordiazepoxide, was discovered in 1955. Benzodiazepines can enhance the effect of the neurotransmitter gamma-aminobutyric acid (GABA) at the GABA_{A} receptor, and can result in sedative, hypnotic (sleep-inducing), anxiolytic (anti-anxiety), anticonvulsant, or muscle relaxant properties. Benzodiazepines are categorized as either short-, intermediate- or long-acting. Exemplary benzodiazapines include alprazolam, bretazenil, bromazepam, brotizolam, chloridazepoxide, cinolazepam, clonazepam, chorazepate, clotiazepam, cloxazolam, delorazepam, diazepam, estazolam, etizolam, ethyl loflazepate, flunitrazepam, flurazepam, flutoprazepam, halazepam, ketazolam, loprazolam, lorazepam, lormetazepam, medazepam, midazolam, nimetazepam, nitrazepam, nordazepam, oxazepam, phenazepam, pinazepam, prazepam, premazepam, pyrazolam, quazepam, temazepam, tatrazepam, and triazolam.

A commonly used anesthetic agent is midazolam. In some embodiments, the benzodiazepine is midazolam.

### Barbiturates

Barbiturates are drugs that have been used as CNS depressants and used e.g., to induce mild sedation, total anesthesia; and used as an anxiolytic, hypnotic, anticonvulsant, analgesic. Side effects of barbiturates include addiction potential, e.g., physical and psychological addiction. Barbiturates may be classified as e.g., ultrashort-acting, short/intermediate-acting, and long-acting. Exemplary barbiturates include pentobarbital, allobarbital, amobarbital, aprobarbital, barbital, brallobarbital.

### Propofol

Propofol (2,6-diisopropylphenol) is a drug, administered intravenously, that provides loss of awareness and can be used with other general anesthetic agents. The main advantages are favorable operating conditions and rapid recovery, but have disadvantages that include a relatively high incidence of apnea and blood pressure reductions.

As used herein, “pharmaceutically acceptable salts” refer to derivatives of the disclosed compounds wherein the parent compound is modified by making the acid-addition or base-addition salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “stereoisomers” refers to compounds made up of the same atoms having the same bond order but having different three-dimensional arrangements of atoms which are not interchangeable. The three-dimensional structures are called configurations. As used herein, the term “enantiomers” refers to two stereoisomers which are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

### Dosage and Pharmacokinetics

The compositions described herein include a therapeutically effective amount of allopregnanolone, and a therapeutically effective amount of a benzodiazepine or anesthetic/sedative. In some embodiments, the allopregnanolone and benzodiazepine or anesthetic/sedative are co-formulated into a single composition or dosage. In some embodiments, the allopregnanolone and benzodiazepine or anesthetic/sedative are administered separately. In some embodiments, the allopregnanolone and benzodiazepine or anesthetic/sedative are administered sequentially. In some embodiments, the allopregnanolone and benzodiazepine or anesthetic/sedative are administered separately and sequentially. In general, at least one of the allopregnanolone and benzodiazepine or anesthetic/sedative is administered parenterally (e.g., intranasally, buccally, intravenously or intramuscularly, for example, intramuscular (IM) injection or intravenously). In some embodiments, both the allopregnanolone and benzodiazepine or anesthetic/sedative is administered parenterally (e.g., intranasally, buccally, intravenously or intramuscularly).

In one embodiment, the allopregnanolone and/or the benzodiazepine or anesthetic/sedative is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 10 μg to about 10 mg per kg of body weight, from about 100 μg to about 5 mg per kg of body weight, from about 250 μg to about 3 mg per kg of body weight, from about 500 μg to about 2 mg per kg of body weight, from about 1 μg to about 50 mg per kg of body weight, from about 1 μg to about 500 μg per kg of body weight; and from about 1 μg to about 50 μg per kg of body weight. Alternatively, the amount of allopregnanolone and/or the benzodiazepine or anesthetic/sedative administered to achieve a therapeutic effective dose is about 0.1 ng, 1 ng, 10 ng, 100 ng, 1 μg, 10 μg, 100 μg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

In one embodiment, the allopregnanolone is administered as an intravenous bolus infusion in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 10 μg to about 5 mg per kg of body weight, from about 100 μg to about 500 μg per kg of body weight, from about 100 μg to about 400 μg per kg of body weight, from about 150 μg to about 350 μg per kg of body weight, from about 250 μg to about 300 μg per kg of body weight. In one embodiment, the allopregnanolone is administered as an intravenous bolus infusion in a dose equivalent to parenteral administration of about 100 to about 400 μg/kg. In some embodiments, the allopregnanolone is administered as an intravenous bolus infusion at about 150 to about 350 μg/kg. In some embodiments, the allopregnanolone is administered as an intravenous bolus infusion at about 250 to about 300 μg/kg. In specific embodiments, the allopregnanolone is administered as an intravenous bolus infusion in a dose equivalent to about 100 μg/kg, 125 μg/kg, 150 μg/kg, 175 μg/kg, 200 μg/kg, 225 μg/kg, 250 μg/kg, 260 μg/kg, 270 μg/kg, 280 μg/kg, 290 μg/kg, 300 μg/kg, 325 μg/kg, or 350 μg/kg.

In one embodiment, the allopregnanolone is administered as an intravenous bolus infusion in a dose equivalent to parenteral administration of about 0.1 nmoles/L to about 100 μmoles/L per kg of body weight, about 1 nmoles/L to about 10 μmoles/L per kg of body weight, about 10 nmoles/L to about 10 μmoles/L per kg of body weight, about 100 nmoles/L to about 10 μmoles/L per kg of body weight, about 300 nmoles/L to about 5 μmoles/L per kg of body weight, about 500 nmoles/L to about 5 μmoles/L per kg of body weight, and about 750 nmoles/L to about 1 μmoles/L per kg of body weight, Alternatively, the amount of allopregnanolone administered to achieve a therapeutic effective dose is about 0.1 ng, 1 ng, 10 ng, 100 ng, 1 μg, 10 μg, 100 μg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 40 mg, 50 mg, 60 mg, 70 mg, 80 mg, 90 mg, 100 mg, 500 mg per kg of body weight or greater.

In some embodiments, the allopregnanolone and/or the benzodiazepine or anesthetic/sedative may be administered once or several times a day. A duration of treatment may follow, for example, once per day for a period of about 1, 2, 3, 4, 5, 6, 7 days or more. In some embodiments, either a single dose in the form of an individual dosage unit or several smaller dosage units or by multiple administration of subdivided dosages at certain intervals is administered. For instance, a dosage unit can be administered from about 0 hours to about 1 hr, about 1 hr to about 24 hr, about 1 to about 72 hours, about 1 to about 120 hours, or about 24 hours to at least about 120 hours post injury. Alternatively, the dosage unit can be administered from about 0.5, 1, 1.5, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours or longer post injury. Subsequent dosage units can be administered any time following the initial administration such that a therapeutic effect is achieved. For instance, additional dosage units can be administered to protect the subject from the secondary wave of edema that may occur over the first several days post-injury.

Area under the curve (AUC) refers to the area under the curve that tracks the serum concentration (nmol/L) of allopregnanolone and/or the benzodiazepine or anesthetic/sedative over a given time following the IV administration of the reference allopregnanolone or the benzodiazepine or anesthetic/sedative standard. By “reference allopregnanolone” or “benzodiazepine standard” or “anesthetic/sedative standard” is intended the formulation of allopregnanolone or the benzodiazepine or anesthetic/sedative that serves as the basis for determination of the total hourly allopregnanolone and/or the benzodiazepine or anesthetic/sedative dose to be administered to a human subject with epilepsy or status epilepticus to achieve the desired positive effect, i.e., a positive therapeutic response that is improved with respect to that observed without administration of allopregnanolone and/or the benzodiazepine or anesthetic/sedative. In an embodiment, the dose of allopregnanolone and/or the benzodiazepine or anesthetic/sedative to be administered provides a final serum level of allopregnanolone and/or the benzodiazepine or anesthetic/sedative of about 100 ng/mL to about 1000 ng/mL, about 1100 ng/mL to about 1450 ng/mL, 100 ng/mL to about 250 ng/mL, about 200 ng/mL to about 350 ng/mL, about 300 ng/mL to about 450 ng/mL, about 350 ng/mL to about 450 ng/mL, about 400 ng/mL to about 550 ng/mL, about 500 ng/mL to about 650 ng/mL, about 600 ng/mL to about 750 ng/mL, about 700 ng/mL to about 850 ng/mL, about 800 ng/mL to about 950 ng/mL, about 900 ng/mL to about 1050 ng/mL, about 1000 ng/mL to about 1150 ng/mL, about 100 ng/mL to about 1250 ng/mL, about 1200 ng/mL to about 1350 ng/mL, about 1300 ng/mL to about 1500 ng/m. In specific embodiments, the serum level of allopregnanolone and/or the benzodiazepine or anesthetic/sedative is about 100 ng/mL, 250 ng/mL, 300 ng/mL, 350 ng/mL, 360 ng/mL, 370 ng/mL, 380 ng/mL, 390 ng/mL, 400 ng/mL, 410 ng/mL, 420 ng/mL, 430 ng/mL, 440 ng/mL, 450 ng/mL, 500 ng/mL, 750 ng/mL, 900 ng/mL, 1200 ng/mL, 1400 ng/mL, or 1600 ng/mL.

In an embodiment, the dose of allopregnanolone to be administered provides a final serum level of allopregnanolone of about 100 nmoles/L to about 5000 nmoles/L, about 100 nmoles/L to about 2500 nmoles/L, about 100 nmoles/L to about 1000 nmoles/L, 100 nmoles/L to about 500 nmoles/L, about 100 nmoles/L to about 250 nmoles/L, about 100 nmoles/L to about 200 nmoles/L, about 125 nmoles/L to about 175 nmoles/L. or about 140 nmoles/L to about 160 nmoles/L. In specific embodiments, the serum level of allopregnanolone and/or the benzodiazepine or anesthetic/sedative is about 100 nmoles/L, 125 nmoles/L, 150 nmoles/L, 175 nmoles/L, 200 nmoles/L, 250 nmoles/L, 300 nmoles/L, 350 nmoles/L, 500 nmoles/L, 750 nmoles/L, 1000 nmoles/L, 1500 nmoles/L, 2000 nmoles/L, 2500 nmoles/L, or 5000 nmoles/L.

In some embodiments, the allopregnanolone and the benzodiazepine or anesthetic/sedative administration includes a time period in which the administration of the benzodiazepine therapy or anesthetic/sedative is weaned off.

As used herein, “weaning” or “weaning dose” refers to an administration protocol which reduces the dose of administration to the patient and thereby produces a gradual reduction and eventual elimination of the benzodiazepine or anesthetic/sedative, either over a fixed period of time or a time determined empirically by a physician's assessment based on regular monitoring of a therapeutic response of a subject. The period of the weaned benzodiazepine or anesthetic/sedative administration can be about 12, 24, 36, 48 hours or longer. Alternatively, the period of the weaned benzodiazepine or anesthetic/sedative administration can range from about 1 to 12 hours, about 12 to about 48 hours, or about 24 to about 36 hours. In some embodiments, the period of the weaned benzodiazepine or anesthetic/sedative administration is about 24 hours.

The weaning employed could be a “linear” weaning. For example, a “10%” linear weaning from 500 mg would go 500, 450, 400, 350, 300, 250, 200, 150, 100, 50. Alternatively, an exponential weaning could be employed which, if the program outlined above is used as an example, the exponential weaning would be, e.g., 500, 450, 405, 365, 329, 296, 266, 239, etc. Accordingly, about a 5%, 10%, 15%, 20%, 25%, 30%, 35%, or 40% linear or exponential weaning could be employed in the methods of the invention. In addition, a linear or exponential weaning of about 1% to 5%, about 6% to 10%, about 11% to 15%, about 16% to 20%, about 21% to 25%, about 26% to 30%, about 31% to 35%, about 36% to 40% could be employed.

In other embodiments, the allopregnanolone and the benzodiazepine or anesthetic/sedative administration includes a final time period in which the administration of allopregnanolone is tapered off.

As used herein, “tapered administration”, “tapered dose”, and “downward taper dose” refers to an administration protocol which reduces the dose of administration to the patient and thereby produces a gradual reduction and eventual elimination of allopregnanolone, either over a fixed period of time or a time determined empirically by a physician's assessment based on regular monitoring of a therapeutic response of a subject. The period of the tapered allopregnanolone administration can be about 12, 24, 36, 48 hours or longer. Alternatively, the period of the tapered allopregnanolone administration can range from about 1 to 12 hours, about 12 to about 48 hours, or about 24 to about 36 hours. In some embodiments, the period of the tapered allopregnanolone administration is about 24 hours.

The drug taper employed could be a “linear” taper. For example, a “10%” linear taper from 500 mg would go 500, 450, 400, 350, 300, 250, 200, 150, 100, 50. Alternatively, an exponential taper could be employed which, if the program outlined above is used as an example, the exponential taper would be, e.g., 500, 450, 405, 365, 329, 296, 266, 239, etc. Accordingly, about a 5%, 10%, 15%, 20%, 25%, 30%, 35%, or 40% linear or exponential taper could be employed in the methods of the invention. In addition, a linear or exponential taper of about 1% to 5%, about 6% to 10%, about 11% to 15%, about 16% to 20%, about 21% to 25%, about 26% to 30%, about 31% to 35%, about 36% to 40% could be employed. In some embodiments, the drug taper is a about 25% linear taper.

Where a subject undergoing therapy exhibits a partial response, or a relapse following completion of the first cycle of the therapy, subsequent courses of allopregnanolone therapy may be needed to achieve a partial or complete therapeutic response. Thus, subsequent to a period of time off from a first treatment period, which may have included a constant allopregnanolone dosing regimen or a two-level allopregnanolone and/or the benzodiazepine or anesthetic/sedative dosing regimen, a subject may receive one or more additional treatment periods including either constant or two-level allopregnanolone and/or the benzodiazepine or anesthetic/sedative dosing regimens. Such a period of time off between treatment periods is referred to herein as a time period of discontinuance. It is recognized that the length of the time period of discontinuance is dependent upon the degree of subject response (i.e., complete versus partial) achieved with any prior treatment periods of the allopregnanolone.

In some embodiments, the allopregnanolone is administered as an intravenous infusion for about 5 minutes to about 1 week; about 30 minutes to about 24 hours, about 1 hour to about 12 hours, about 2 hours to about 12 hours, about 4 hours to about 12 hours, about 6 hours to about 12 hours, about 6 hours to about 10 hours; about 5 minutes to about 1 hour, about 5 minutes to about 30 minutes; about 12 hours to about 1 week, about 24 hours to about 1 week, about 2 days to about 5 days, or about 3 days to about 5 days. In one embodiment, the allopregnanolone is administered as an intravenous infusion for about 5, 10, 15, 30, 45, or 60 minutes or longer; about 1, 2, 4, 6, 8, 10, 12, 16, or 24 hours or longer; about 1, 2, 3, 4, 5, 6, 7, 8, 9, or 10 days or longer.

These multiple treatment sessions are referred to herein as maintenance cycles, where each maintenance cycle includes a completed constant or two-level allopregnanolone or the benzodiazapine dosing regimen. By “completed two-level progesterone, allopregnanolone, or a synthetic progestin dosing regimen” is intended the subject has been administered both the first period and the second period of allopregnanolone or the benzodiazapine dosing. The necessity for multiple maintenance cycles can be assessed by monitoring the physiological and behavioral improvement of the patient. The duration between maintenance cycles can be about 1 hr, 15 hr, 1 day, 2 day, 3 day, 4 day, 5 day, 6 day or other such time periods falling within the range of about 1 day to about 14 days.

In some embodiments, the maintenance cycle is about 2 days. In some embodiments, the maintenance cycle is about 3 days. In some embodiments, the maintenance cycle is about 4 days. In some embodiments, the maintenance cycle is about 5 days.

In some embodiments, the maintenance cycle begins from about 30 minutes to about 24 hours, about 30 minutes to about 12 hours, about 30 minutes to about 8 hours, about 30 minutes to about 4 hours, about 30 minutes to about 2 hours, about 30 minutes to about 1 hour following the initial bolus infusion administration. In some embodiments, the maintenance cycle begins 30 minutes, 45 minutes, 1 hour, 2 hours, 3 hours, 4 hours, 6 hours, 8 hours, 12 hours, 24 hours, or longer following the initial bolus infusion administration.

In one embodiment, the maintenance cycle the allopregnanolone is administered as an intravenous infusion at an amount of allopregnanolone/unit time of about 20 to about 5000 μg/kg/hr. In some embodiments, the maintenance cycle the allopregnanolone is administered as an intravenous infusion at an amount of allopregnanolone/unit time of about 20 to about 2500 μg/kg/hr. In some embodiments, the maintenance cycle the allopregnanolone is administered as an intravenous infusion at an amount of allopregnanolone/unit time of about 20 to about 500 μg/kg/hr. In some embodiments, the allopregnanolone is administered as an intravenous infusion at a rate of about 20 to about 250 μg/kg/hr. In some embodiments, the allopregnanolone is administered as an intravenous infusion at an amount of allopregnanolone/unit time of about 20 to about 200 μg/kg/hr. In some embodiments, the allopregnanolone is administered as an intravenous infusion at an amount of allopregnanolone/unit time of about 20 to about 150 μg/kg/hr. In some embodiments, the allopregnanolone is administered as an intravenous infusion at an amount of allopregnanolone/unit time of about 50 to about 100 μg/kg/hr. In some embodiments, the allopregnanolone is administered as an intravenous infusion at an amount of allopregnanolone/unit time of about 70 to about 100 μg/kg/hr. In specific embodiments, the allopregnanolone is administered as an intravenous infusion at an amount of allopregnanolone/unit time of about 25 μg/kg/hr, 50 μg/kg/hr, 75 μg/kg/hr, 80 μg/kg/hr, 85 μg/kg/hr, 86 μg/kg/hr, 87 μg/kg/hr, 88 μg/kg/hr, 89 μg/kg/hr, 90 μg/kg/hr, 100 μg/kg/hr, 125 μg/kg/hr, 150 μg/kg/hr, or 200 μg/kg/hr.

In one embodiment, the allopregnanolone is administered as an intravenous infusion in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 10 μg to about 5 mg per kg of body weight; and from about 100 μg to about 1000 μg per kg of body weight. In one embodiment, the allopregnanolone is administered as an intravenous infusion in a dose equivalent to parenteral administration of about 0.1 nmoles/L to about 100 μmoles/L per kg of body weight, about 1 nmoles/L to about 10 μmoles/L per kg of body weight, about 10 nmoles/L to about 10 μmoles/L per kg of body weight, about 100 nmoles/L to about 10 μmoles/L per kg of body weight, about 300 nmoles/L to about 5 μmoles/L per kg of body weight, about 500 nmoles/L to about 5 μmoles/L per kg of body weight, and about 750 nmoles/L to about 5 μmoles/L per kg of body weight, Alternatively, the amount of allopregnanolone administered to achieve a therapeutic effective dose is about 0.1 ng, 1 ng, 10 ng, 100 ng, 1 μg, 10 μg, 100 μg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 40 mg, 50 mg, 60 mg, 70 mg, 80 mg, 90 mg, 100 mg, 500 mg per kg of body weight or greater. As used herein, “about” means approximately plus or minus ten percent.

### Formulations

Formulations described herein include allopregnanolone and/or a benzodiazepine or anesthetic/sedative in combination with one or more pharmaceutically acceptable excipients. In some embodiments, a formulation includes both allopregnanlone and a benzodiazepine or anesthetic/sedative.

### Solubilization of Allopregnanolones

The lipophilic nature of allopregnanolone can make it different to formulate for in vivo administration. As discussed above, allopregnanolone can be formulated with a host, such as a cyclodextrin to improve the solubility. Alternatively, or additionally, allopregnanolone can be modified in an attempt to improve the solubility. For example, polar groups can be introduced onto position 16α with the goal of increasing water solubility, brain accessibility, and potency of allopregnanolones as described in Kasal et al., J. Med. Chem., 52(7), 2119-215 (2009). In order to provide formulations capable of delivering therapeutically effective dosages, a variety of methods can be employed to enhance the solubility and bioavailability of allopregnanolones. See, for example, “Water-Insoluble Drug Formulation”, 2nd Edition, edited by Rong Liu (CRC Press, Boca Raton, Fla., 2008). Using the techniques described below, a solubilized formulation of one or more allopregnanolones can be prepared. These solubilized formulations can be further incorporated into parenteral and non-parenteral formulations.

### Inclusion Complexes

The solubility of a compound such as allopregnanolone can be improved by inclusion complexation (e.g., host-guest formulations). Inclusion complexes are formed when a nonpolar molecule (i.e., the guest, such as a drug with poor aqueous stability) or portion of a molecule inserts into a nonpolar cavity of another molecule or group of molecules (i.e., the host). If the host molecule or molecules exhibit water good solubility, the solubility of the host-guest complex will be greater than the solubility of the guest alone.

Dextrans are soluble polysaccharides produced by bacteria and yeasts. They are characterized by a predominance (>95%) of α(1-6) backbone linkages and varying proportions of α(1-2), α(1-3) and α(1-4) linkages typically at branch points 1, 2. Dextrins are partially hydrolyzed glucose homopolymers composed exclusively of α(1-4) backbone linkages.

Cyclodextrins are cyclic oligosaccharides containing or comprising six (α-cyclodextrin), seven (β-cyclodextrin), eight (γ-cyclodextrin), or more α-(1,4)-linked glucose residues. The hydroxyl groups of the cyclodextrins are oriented to the outside of the ring while the glucosidic oxygen and two rings of the non-exchangeable hydrogen atoms are directed towards the interior of the cavity. As a result, cyclodextrins possess a hydrophobic inner cavity combined with a hydrophilic exterior which conveys water solubility. Upon combination with a hydrophobic drug, such as allopregnanolone, the allopregnanolone (i.e., the guest) inserts into the hydrophobic interior of the cyclodextrin (i.e., the host). The host-guest complex retains water solubility as a consequence of the hydrophobic exterior of the cyclodextrin ring.

Allopregnanolone-cyclodextrin complexes can, as solubility permits, be incorporated into any of the parenteral and non-parenteral formulations described below. If desired, the aqueous solubility of solid neuoractive steroid-cyclodextrin complexes can be further enhanced by isolating the neuoractive steroid-cyclodextrin complex as a solid via lyophilization and/or via micronizing the solid allopregnanolone-cyclodextrin complex.

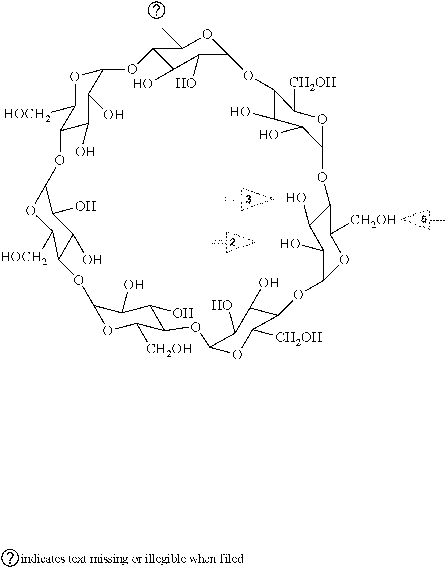

This cyclic orientation provides a truncated cone structure that is hydrophilic on the exterior and lipophilic on the interior. Cyclodextrin complexes are formed when a guest molecule is partially or fully contained in the interior of the cavity. The parent α-, β-, and γ-cyclodextrins (particularly β) have limited aqueous solubility and show toxicity when given parenterally. Therefore, the parent cyclodextrin structure can be chemically modified to generate a parenterally safe CD-derivative. The modifications are typically made at one or more of the 2, 3, or 6 position hydroxyls.

Allopregnanolone-cyclodextrin complexes are preferably formed from a cyclodextrin selected from the group consisting of α-cyclodextrin, β-cyclodextrin, γ-cyclodextrin, and derivatives thereof. The cyclodextrin may be chemically modified such that some or all of the primary or secondary hydroxyl groups of the macrocycle, or both, are functionalized with a pendant group. Suitable pendant groups include, but are not limited to, sulfinyl, sulfonyl, phosphate, acyl, and C₁-C₁₂ alkyl groups optionally substituted with one or more (e.g., 1, 2, 3, or 4) hydroxy, carboxy, carbonyl, acyl, oxy, oxo; or a combination thereof. Methods of modifying these alcohol residues are known in the art, and many cyclodextrin derivatives are commercially available, including sulfo butyl ether β-cyclodextrins available under the trade name CAPTISOL® from Ligand Pharmaceuticals (La Jolla, Calif.).

Examples of suitable cyclodextrins for use in allopregnanolone formulations, can include cyclodextrins disclosed in U.S. Pat. Nos. 5,874,418; 6,046,177; and 7,635,733, which are herein incorporated by reference. Other examples of suitable cyclodextrins for use in allopregnanolone formulations non-exclusively include α-cyclodextrin; β-cyclodextrin; γ-cyclodextrin; methyl α-cyclodextrin; methyl β-cyclodextrin; methyl γ-cyclodextrin; ethyl β-cyclodextrin; butyl α-cyclodextrin; butyl β-cyclodextrin; butyl γ-cyclodextrin; pentyl γ-cyclodextrin; hydroxyethyl β-cyclodextrin; hydroxyethyl γ-cyclodextrin; 2-hydroxypropyl α-cyclodextrin; 2-hydroxypropyl β-cyclodextrin; 2-hydroxypropyl γ-cyclodextrin; 2-hydroxybutyl β-cyclodextrin; acetyl α-cyclodextrin; acetyl β-cyclodextrin; acetyl γ-cyclodextrin; propionyl β-cyclodextrin; butyryl β-cyclodextrin; succinyl α-cyclodextrin; succinyl τ-cyclodextrin; succinyl γ-cyclodextrin; benzoyl β-cyclodextrin; palmityl β-cyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl β-cyclodextrin; phosphate ester α-cyclodextrin; phosphate ester β-cyclodextrin; phosphate ester γ-cyclodextrin; β-trimethylammonium-2-hydroxypropyl β-cyclodextrin; sulfobutyl ether β-cyclodextrin; carboxymethyl α-cyclodextrin; carboxymethyl β-cyclodextrin; carboxymethyl γ-cyclodextrin, and combinations thereof.

Preferred cyclodextrins include, but are not limited to, alkyl cyclodextrins, hydroxy alkyl cyclodextrins, such as hydroxy propyl β-cyclodextrin, carboxy alkyl cyclodextrins and sulfoalkyl ether cyclodextrins, such as sulfo butyl ether β-cyclodextrin.

In particular embodiments, the cyclodextrin is a alpha, beta, or gamma cyclodextrin having a plurality of charges (e.g., negative or positive) on the surface. In more particular embodiments, the cyclodextrin is a β-cyclodextrin containing or comprising a plurality of functional groups that are negatively charged at physiological pH. Examples of such functional groups include, but are not limited to, carboxylic acid (carboxylate) groups, sulfonate (RSO₃⁻), phosphonate groups, phosphinate groups, and amino acids that are negatively charged at physiological pH. The charged functional groups can be bound directly to the cyclodextrins or can be linked by a spacer, such as an alkylene chain. The number of carbon atoms in the alkylene chain can be varied, but is generally between about 1 and 10 carbons, preferably 1-6 carbons, more preferably 1-4 carbons. Highly sulfated cyclodextrins are described in U.S. Pat. No. 6,316,613.

In one embodiment, the cyclodextrins is a β-cyclodextrin functionalized with a plurality of sulfobutyl ether groups. Such a cyclodextrins is sold under the trade name CAPTISOL®.

CAPTISOL® is a polyanionic beta-cyclodextrin derivative with a sodium sulfonate salt separated from the lipophilic cavity by a butyl ether spacer group, or sulfobutylether (SBE). CAPTISOL® is not a single chemical species, but comprised of a multitude of polymeric structures of varying degrees of substitution and positional/regional isomers dictated and controlled to a uniform pattern by a patented manufacturing process consistently practiced and improved to control impurities.

CAPTISOL® contains six to seven sulfobutyl ether groups per cyclodextrin molecule. Because of the very low pKa of the sulfonic acid groups, CAPTISOL® carries multiple negative charges at physiologically compatible pH values. The four-carbon butyl chain coupled with repulsion of the end group negative charges allows for an “extension” of the cyclodextrin cavity. This often results in stronger binding to drug candidates than can be achieved using other modified cyclodextrins. It also provides a potential for ionic charge interactions between the cyclodextrin and a positively charged drug molecule. In addition, these derivatives impart exceptional solubility and parenteral safety to the molecule. Relative to beta-cyclodextrin, CAPTISOL® provides higher interaction characteristics and superior water solubility in excess of 100 grams/100 ml, a 50-fold improvement.

In other embodiments, the cyclodextrins has plurality of functional groups that are negatively charged at physiological pH. Suitable positively charged groups include, but are not limited to, quaternary ammonium groups. Exemplary cyclodextrins include, but are not limited to, mono-6(A)-butylammonium-6(A)-deoxy-beta-cyclodextrin tosylate (BuAM-beta-CD) and Amine- and guanidine-derivatised β-cyclodextrin (βCD).

Preferably, the cyclodextrin is present in an amount of from about 0.1% to about 40% w/w of the overall formulation, preferably from about 5% to about 40% w/w, more preferably about 10% to about 40% w/w, most preferably about 10% to about 35% w/w. In some embodiments, the cyclodextrin is present in an amount of about 25% w/w of the overall formulation. In certain embodiments, the concentration of the cyclodextrins is from about 15% to about 35% w/w, preferably from about 20% to about 35% w/w, more preferably about 30% to about 35% w/w. In one embodiment, the formulation contains about 1 to about 2, preferably about 1.5 mg allopregnanolone per mL of cyclodextrin, e.g., CAPTISOL®. In one embodiment, the formulation contains about 5 mg allopregnanolone per mL of cyclodextrin, e.g., CAPTISOL®.

### Formulations for Parenteral Administration

The compounds (e.g., allopregnanolone) described herein can be formulated for parenteral administration. Preferred doses, dosage forms, or modes of administration are parenteral, e.g., intranasally, buccally, intravenous, intramuscular, subcutaneous, intraparenteral, bucosal, sublingual, intraocular, and topical (e.g., intravenous or intramuscular). In another embodiment, the informational material can include instructions to administer the compound described herein to a suitable subject, e.g., a human, e.g., a human having or at risk for a disorder described herein. In some preferred embodiments, at least one of the allopregnanolone and benzodiazepine or anesthetic/sedative is formulated for parenteral administration. In some embodiments, both the allopregnanolone and the benzodiazepine or anesthetic/sedative are formulated for parenteral administration.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

In some embodiments, the parenteral formulations are prepared as an injectable formulation, e.g., for intravenous administration. In some embodiments, the parenteral formulation comprises a compound (e.g., allopregnanolone), and a cyclodextrin, e.g., a β-cyclodextrin, e.g., a sulfo butyl ether β-cyclodextrin, e.g., CAPTISOL®). In some embodiments, the parenteral formulation comprises allopregnanolone and a sulfo butyl ether β-cyclodextrin, e.g., CAPTISOL®.

The carrier can be a solvent or dispersion medium containing or comprising, for example, water (e.g., Water for Injection, USP), ethanol, one or more polyols (e.g., glycerol, propylene glycol, and liquid polyethylene glycol), oils, such as vegetable oils (e.g., peanut oil, corn oil, sesame oil, etc.), and combinations thereof.

The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing or comprising carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

In some embodiments, at least one of the allopregnanolone or the benzodiazepine or anesthetic/sedative is formulated for intranasal, buccal, intramuscular or intravenous administration (e.g., intramuscular or intravenous administration). In some embodiments, both of the allopregnanolone and the benzodiazepine or anesthetic/sedative are formulated for intranasal, buccal, intramuscular or intravenous administration (e.g., intramuscular or intravenous administration).

The parenteral formulations described herein can be formulated for controlled release including immediate release, delayed release, extended release, pulsatile release, and combinations thereof.

### Nano- and Microparticles

For parenteral administration, the compounds, and optionally one or more additional active agents, can be incorporated into microparticles, nanoparticles, or combinations thereof that provide controlled release. In embodiments wherein the formulations contains two or more drugs, the drugs can be formulated for the same type of controlled release (e.g., delayed, extended, immediate, or pulsatile) or the drugs can be independently formulated for different types of release (e g, immediate and delayed, immediate and extended, delayed and extended, delayed and pulsatile, etc.).

For example, the compounds and/or one or more additional active agents can be incorporated into polymeric microparticles which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers which are slowly soluble and form a gel in an aqueous environment, such as hydroxypropyl methylcellulose or polyethylene oxide may also be suitable as materials for drug containing or comprising microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins which are water insoluble, such as zein, can also be used as materials for the formation of drug containing or comprising microparticles. Additionally, proteins, polysaccharides and combinations thereof which are water soluble can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug containing or comprising microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. Detailed descriptions of these processes can be found in “Remington—The science and practice of pharmacy”, 20th Edition, Jennaro et. al., (Phila, Lippencott, Williams, and Wilkens, 2000).

For some carrier materials it may be desirable to use a solvent evaporation technique to produce drug containing or comprising microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing or comprising microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin (Cortesi, R., et al., Biomaterials 19 (1998) 1641-1649). Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing or comprising microparticles or drug particles, a water soluble protein can be spray coated onto the microparticles and subsequently cross-linked by the one of the methods described above. Alternatively, drug containing or comprising microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

In some embodiments, at least one of the allopregnanolone and/or the benzodiazepine or anesthetic/sedative is formulated for intranasal, buccal, intramuscular or intravenous administration (e.g., intramuscular or intravenous administration). In some embodiments, both of the allopregnanolone and the benzodiazepine or anesthetic/sedative are formulated for intranasal, buccal, intramuscular or intravenous administration (e.g., intramuscular or intravenous administration).

The compounds described herein can be formulated for depot injection. In a depot injection, the active agent is formulated with one or more pharmaceutically acceptable carriers that provide for the gradual release of active agent over a period of hours or days after injection. The depot formulation can be administered by any suitable means; however, the depot formulation is typically administered via subcutaneous or intramuscular injection.

A variety of carriers may be incorporated into the depot formulation to provide for the controlled release of the active agent. In some cases, depot formulations contain one or more biodegradable polymeric or oligomeric carriers. Suitable polymeric carriers include, but are not limited to poly(lactic acid) (PLA), poly(lactic-co-glycolic acid) (PLGA), poly(lactic acid)-polyethyleneglycol (PLA-PEG) block copolymers, polyanhydrides, poly(ester anhydrides), polyglycolide (PGA), poly-3-hydroxybutyrate (PHB) and copolymers thereof, poly-4-hydroxybutyrate (P4HB), polycaprolactone, cellulose, hydroxypropyl methylcellulose, ethylcellulose, as well as blends, derivatives, copolymers, and combinations thereof.

In depot formulations containing or comprising a polymeric or oligomeric carrier, the carrier and active agent can be formulated as a solution, an emulsion, or suspension. One or more allopregnanolones, and optionally one or more additional active agents, can also be incorporated into polymeric or oligomeric microparticles, nanoparticles, or combinations thereof.

In some cases, the formulation is fluid and designed to solidify or gel (i.e., forming a hydrogel or organogel) upon injection. This can result from a change in solubility of the composition upon injection, or for example, by injecting a pre-polymer mixed with an initiator and/or cross-linking agent. The polymer matrix, polymer solution, or polymeric particles entrap the active agent at the injection site. As the polymeric carrier is gradually degraded, the active agent is released, either by diffusion of the agent out of the matrix and/or dissipation of the matrix as it is absorbed. The release rate of the active agent from the injection site can be controlled by varying, for example, the chemical composition, molecular weight, crosslink density, and concentration of the polymeric carrier. Examples of such systems include those described in U.S. Pat. Nos. 4,938,763, 5,480,656 and 6,113,943.

Depot formulations can also be prepared by using other rate-controlling excipients, including hydrophobic materials, including acceptable oils (e.g., peanut oil, corn oil, sesame oil, cottonseed oil, etc.) and phospholipids, ion-exchange resins, and sparingly soluble carriers.

The depot formulation can further contain a solvent or dispersion medium containing or comprising, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of allopregnanolone or a benzodiazepine or anesthetic/sedative as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing or comprising carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

In some embodiments, at least one of the allopregnanolone and/or the benzodiazepine or anesthetic/sedative is formulated for intranasal, buccal, intramuscular or intravenous administration (e.g., intramuscular or intravenous administration). In some embodiments, both of the allopregnanolone and the benzodiazepine or anesthetic/sedative are formulated for intranasal, buccal, intramuscular or intravenous administration (e.g., intramuscular or intravenous administration).

Combinations with Active Compounds

A composition described herein can be administered adjunctively with other active compounds such as anesthetics or sedatives, e.g., benzodiazepine, e.g., midazalm, propofol, pentobarbital, and ketamine

### Methods of Use

A composition described herein can be administered to a subject in need thereof to treat a disorder described herein, wherein the disorder described herein is related to a preceding structural condition in the brain of said subject. Exemplary disorders include epilepsy, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; a seizure, e.g., acute repetitive seizures, cluster seizures.

In one aspect, provided herein is a method of treating a subject having a seizure-related disorder, wherein said seizure-related disorder is preceded by a condition related to a structural modification in the brain of said subject, the method comprising: administering to said subject, an effective amount of allopregnanolone, thereby treating said subject. In some embodiments, the structural modification is a lesion in the brain that is visible using standard imaging techniques, e.g., magnetic resonance imaging (MRI) or a computerized tomography (CT) scan. In some embodiments, the the epilepsy or status epilepticus is related to, e.g., caused by, the condition related to a structural modification in the brain of said subject. In some embodiments, the condition associated with the structural modification is a tumor (e.g., the tumor is a source of the structural modification). In some embodiments, the structural modification is a vascular structural modification. In some embodiments, the condition related to a structural modification is a brain aneurysm with associated hemorrhage. In some embodiments, the condition related to a structural modification is a intraparenchymal hemorrhage. In some embodiments, the condition related to a structural modification is a brain arteriovenous malformation with associated hemorrhage. In some embodiments, the condition related to a structural modification is an ischemic stroke. In some embodiments, the condition related to a structural modification is a focal cortical dysplasias or malformation of cortical development. In some embodiments, the condition related to a structural modification is an intraparenchymal brain tumor. In some embodiments, the condition related to a structural modification is post-traumatic porenenchephaly or gliosis. In some embodiments, the condition related to a structural modification is a cerebral abscess. In some embodiments, the condition related to a structural modification is a central nervous system infection, e.g., neurocysticercosis. In some embodiments, the condition related to a structural modification is encephalitis. In some embodiments, the condition related to a structural modification is multiple sclerosis. In some embodiments, the condition related to a structural modification is a demyelinating lesion.

In another aspect, provided herein is a method of treating a subject having a seizure-related disorder, wherein said seizure-related disorder is preceded by a condition selected from the group consisting of a brain aneurysm with associated hemorrhage, an intraparenchymal hemorrhage, a brain arteriovenous malformation with associated hemorrhage, an ischemic stroke, a focal cortical dysplasias or malformation of cortical development, an intraparenchymal brain tumor, post-traumatic porenenchephaly or gliosis, a cerebral abscess, a central nervous system infection, encephalitis, multiple sclerosis, and a demyelinating lesion, the method comprising: administering to said subject, an effective amount of allopregnanolone, thereby treating said subject.

In some embodiments, a composition described herein (e.g., a composition comprising allopregnanolone), is administered to a subject under general anesthesia.

### Seizures and Seizure-Related Disorders

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures.

Publications cited herein and the materials for which they are cited are specifically incorporated by reference.

### Status Epilepticus (SE)

Status epilepticus (SE) encompasses a group of disorders all involving persistent or recurring seizures. The standard of care in the United States (US) typically involves initially treating status epilepticus with a benzodiazepine as a first line agent for “early” SE. A recent study showed that 26.6% of patients did not respond to first line midazolam intramuscular (IM) treatment and 36.6% of patients did not respond to lorazepam intravenous (IV) treatment (Silbergleit et al, 2012).

If patients continue to have seizures after administration of the benzodiazepine, they are treated with a second-line anti-epileptic drug for “established” SE, which in the US is generally fos-phenytoin IV or phenytoin IV. If patients continue to have seizures after administration of first and second line drugs, they are said to enter into a stage of “refractory” SE (RSE).

The generally accepted course of therapy for patients in RSE is general anesthesia (GA) with an agent such as midazolam, propofol, pentobarbital, or ketamine There is no drug approved for RSE and clinical evidence of the comparative efficacy of the commonly used drugs is lacking (Shorvon, 2011). The goal of therapy with these GA agents is to induce an electroencephalographic “burst suppression” state, in an attempt to block the excitotoxic cerebral damage believed to occur as a result of continued seizure activity in the brain. Burst-suppression is an electroencephalography pattern consisting of alternative periods of slow waves of high amplitude (the burst) and periods of a flat electroencephalogram (EEG) (the suppression); it is associated with comatose states of various etiologies and anesthesia (Amzica & Kroeger, 2011). The goal of a therapy is that when a patient is weaned from the general anesthesia, the patient will no longer have clinical or electrographic seizure activity. EEG and EEG terminology is described in Hirsch et al., J. Clin. Neurophysiol. 2013; 30: 1-27, which reference is incorporated in its entirety.

Patients said to be in super-refractory SE (SRSE) or super-refractory generalized SE are a subgroup of RSE patients who have continued or recurrent seizures 24 hours or more after the onset of anesthetic therapy; it often seen as the recurrence of seizure activity as the patient is weaned from the anesthetic therapy. It has been estimated that ˜15% of patients admitted to hospitals with SE become super-refractory (Shorvon & Ferlisi, 2011).

SE can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

Compositions described herein can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

### Epilepsy

Epilepsy is a brain disorder characterized by repeated seizures over time. Types of epilepsy can include, but are not limited to generalized epilepsy, e.g., childhood absence epilepsy, juvenile nyoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### EXAMPLES

### Example 1: Formulation of SAGE-547 Injection

SAGE-547 injection is a solution of 5 mg/mL allopregnanolone in Sterile Water for Injection (SWFI), USP and 250 mg/mL betadex sulfobutyl-ether sodium, NF. It is further diluted with Sterile Water for Injection, USP (SWFI) in IV bags to achieve an allopregnanolone concentration of approximately 1.67 mg/mL in an approximately isotonic solution and will be administered intravenously.

### Example 2: A Randomized, Double-Blind, Placebo-Controlled Study to Evaluate the Efficacy and Safety of SAGE-547 Injection in the Treatment of Subjects with Super-Refractory Status Epilepticus

### Number of Subjects

The study will randomize 140 subjects at up to 180 sites.

### Study Population

Subjects will be aged two years or more, in SRSE1, and being managed in an intensive care setting.

### Duration of Subject Involvement

Individual subject participation will be up to 30 days.

### Study Design

This is a randomized, double-blind, placebo-controlled trial, designed to evaluate the efficacy and safety of SAGE-547 administered as a continuous intravenous infusion to subjects in SRSE.

Patients will follow one of three paths to entry to the study. The first path is for subjects who are admitted to the intensive care unit at the study site in status epilepticus, having failed first- and second-line therapies, with a view to initiating burst or seizure suppression with a third-line agent. These subjects will be consented for the study after admission to the intensive care unit, before or after any third-line agents are administered for burst or seizure suppression. These subjects are unlikely to be able to consent themselves for the study due to coma or mental incapacity from the status epilepticus, and so consent will be obtained from their legally authorized representative (LAR). Subjects will be administered one or more third-line agents at a dose sufficient to maintain a burst or seizure suppression pattern on the EEG for 24 hours. Once burst or seizure suppression has been maintained for 24 hours, the third-line agent or agents will be weaned. This wean is called the qualifying wean (QW), and subjects who are successfully weaned will be followed for approximately three weeks to collect medication, epilepsy status, adverse event, and outcome data. Subjects who fail the QW will have the same or a different third-line agent regimen re-instituted at doses intended to result in EEG burst or seizure suppression and will be randomized to concomitant SAGE-547 or placebo. Burst suppression must be maintained for the first 12 hours of the infusion of blinded study drug.

The second path to entry to the study is for subjects who have either been transferred from another hospital or from within the study site institution and who are already on a continuous intravenous infusion of thirdline agent with a burst or seizure suppression pattern on the EEG. These subjects may have had one or more previous unsuccessful weans and may be on more than one third-line agent. Appropriate consent will be obtained and the third-line agent will be continued after consent until at least 24 hours of continuous burst or seizure suppression has been achieved. The duration of post-consent third-line agent administration will depend on how long this episode of burst or seizure suppression was maintained prior to consent being obtained. Once at least 24 hours of continuous EEG burst or seizure suppression has been achieved, the third-line agent or agents will be weaned. This is the QW. Subjects who are successfully weaned will be followed for approximately three weeks to collect medication, epilepsy status, adverse event, and outcome data. Subjects who fail the QW will have the same or a different third-line agent regimen re-instituted at doses intended to result in EEG burst or seizure suppression and will be randomized to concomitant SAGE-547 or placebo. Burst suppression must be maintained for the first 12 hours of the infusion of blinded study drug.

The third path to entry to the study is for subjects who have either been transferred from another hospital or from within the study site institution and who arrive at the study site intensive care unit without a burst or seizure suppression pattern on the EEG or not on a continuous infusion of at least one third-line agent.

These subjects may have had one or more previous unsuccessful weans. It is common practice to stop all third-line agents in these subjects to assess the underlying clinical and EEG presentation, while optimizing concomitant AEDs. If the decision is made to re-administer a third-line agent, consent will be obtained from the subject's LAR. The subject will then be administered one or more third-line agent at a dose sufficient to maintain a burst or seizure suppression pattern on the EEG for 24 hours, at which time the third-line agent or agents will be weaned. This is the QW. Subjects who are successfully weaned will be followed for approximately three weeks to collect medication, epilepsy status, adverse event, and outcome data. Subjects who fail the QW will have the same or a different third-line agent regimen re-instituted at doses intended to result in EEG burst or seizure suppression and will be randomized to concomitant SAGE-547 treatment or placebo. Burst suppression must be maintained for the first 12 hours of the infusion of blinded study drug. Once subjects are deemed to be failures of the QW, they must be randomized and the blinded study drug infusion commenced within eight hours of the investigator's determination that they failed the QW.

Subjects will be randomized to SAGE-547 or placebo in a 1:1 ratio with 70 subjects to be randomized to SAGE-547 and 70 subjects to be randomized to placebo. The randomization will be stratified by concomitant pentobarbital/thiopental use (yes or no) and number of previous third-line agent wean attempts prior to randomization (one vs two or more). A dynamic randomization (minimization algorithm) will be used to achieve 1:1 balance across the stratification factors and between the treatment groups (SAGE-547 and placebo). The randomized portion of the study will be blinded, with the two treatments (SAGE-547 and placebo) being indistinguishable so that subjects, relatives, nursing and medical staff, pharmacists and monitors will not be able to ascertain which subject was allocated to which treatment.

For all subjects in the study, attempts will be made to wean the subject off all third-line agents starting at hour (H) 49 of the blinded study treatment infusion, with the aim of completing all weans by hour (H) 120 of the study treatment infusion. Efficacy will be evaluated by applying success criteria, with a success defined as both the ability to wean the subject off all third-line agents before the end of the infusion of blinded study medication without the need to reinstitute a third-line agent for at least 24 hours following cessation of the blinded study medication infusion, and concurrent signs of physiologic brain activity as determined by EEG. Those subjects who fail the primary endpoint, and require re-institution of a third-line agent regimen before the end of the blinded study drug infusion or within 24 hours of completing the blinded study drug infusion will be eligible to receive an open-label infusion of SAGE-547 at a dose higher than that administered in the blinded part of the study. The blind will be maintained for all subjects in the study, so subjects who failed on SAGE-547 and subjects who failed on placebo will all subsequently be eligible to be administered SAGE-547 at the higher dose.

### Study Objectives

### Primary Objective:

To determine the response to a 144-hour (6 day) continuous intravenous infusion of SAGE-547 compared to placebo administered to support the weaning of all third-line agents in adult and pediatric subjects with SRSE, and for the response to endure at least 24 hours after the end of the SAGE-547 or placebo infusion (primary response).

### Secondary Objectives:

1. To compare between SAGE-547 and placebo the time between meeting the primary response endpoint and the re-institution of any third-line agent for seizure or burst suppression up to Visit 12;
2. To compare between SAGE-547 and placebo secondary response, defined as success of weaning the subject off all third-line agents before the end of the first infusion of SAGE-547 or placebo;
3. To compare between SAGE-547 and placebo the time between meeting the secondary response endpoint, defined in (2) above, and the re-institution of any third-line agent for seizure or burst suppression up to Visit 12;
4. To compare the change in the Clinical Global Impression scale (CGI) between SAGE-547 and placebo up to Visit 12;
5. To determine the number of days after the end of the first infusion of study drug that the subject does not have status epilepticus, up to Visit 12;
6. To determine the number of days after the end of the first infusion of study drug that the subject does not have seizures (convulsive and non-convulsive) up to Visit 12;
7. To determine the number of separate episodes of status epilepticus occurring up to Visit 12;
8. To determine the proportion of subjects with a new diagnosis of epilepsy after Visit 11.

### Safety Objectives:

To determine the safety and tolerability of a 144-hour infusion of SAGE-547 in four groups of subjects with SRSE (one infusion of SAGE-547, one infusion of placebo, one infusion of placebo followed by one infusion of SAGE-547, and two infusions of SAGE-547) via:

a. Adverse events and medications;
b. Laboratory testing (hematology, serum chemistry, and urinalysis);
c. Vital signs (blood pressure, heart rate, temperature, and weight);
d. ECG parameters;
e. Mortality.

### Other Objectives:

1. To evaluate the impact of treatment with a higher dose SAGE-547 infusion;
2. To evaluate the pharmacokinetics of SAGE-547, and present Captisol® and identified SAGE-547 metabolite plasma concentrations;
3. To correlate QT/QTc interval with plasma concentrations of SAGE-547;
4. To determine the number of days in the ICU, number of days in hospital, discharge destination from the ICU, discharge destination from the hospital, dischargeability criteria, resource use for subjects discharged from hospital before Visit 12/12R;
5. To evaluate the Full Outline of UnResponsiveness (FOUR) Score;
6. To evaluate the Glasgow Outcome Score (GOS);
7. To evaluate the Supervision Rating Scale (SRS);
8. To evaluate the Modified Rankin Score (mRS) (age ≥17 years).

### Endpoints

### Primary Endpoint:

Success or failure, with success defined as weaning the subject off all third-line agents before completion of the first blinded infusion of SAGE-547 or placebo, and not having to re-institute any third-line agent for seizure or burst suppression during the 24 hours after the end of the first infusion of SAGE-547 or placebo, and concurrent signs of physiologic brain activity as determined by EEG (primary response).

### Secondary Endpoints:

1. The time between meeting the primary response endpoint and the re-institution of any third-line agent for seizure or burst suppression up to Visit 12;
2. Secondary response, defined as success of weaning the subject off all third-line agents before the end of the first SAGE-547 or placebo infusion;
3. The time between meeting the secondary response endpoint, defined in (2) above, and the reinstitution of any third-line agent for seizure or burst suppression up to Visit 12;
4. The assessment of clinical status as measured by change in the CGI from Visit 1 (Screening) to Visit 12;
5. The number of days after the end of the first study drug infusion that the subject does not have status epilepticus, up to Visit 12;
6. The number of days after the end of the first study drug infusion that the subject does not have seizures (convulsive and non-convulsive) up to Visit 12;
7. The number of separate episodes of status epilepticus occurring up to Visit 12;
8. The proportion of subjects with a new diagnosis of epilepsy after Visit 11.

### Safety Endpoints:

1. Adverse events and medications;
2. Laboratory testing (hematology, serum chemistry, and urinalysis);
3. Vital signs (blood pressure, heart rate, temperature, and weight);
4. ECG parameters;

### 5. Mortality.

### Other Endpoints:

1. The same endpoints as described for the primary and secondary endpoints will be calculated for those subjects who are treated with a higher dose of SAGE-547.
2. Standard pharmacokinetic data derived from SAGE-547 plasma concentrations, and presentation of Captisol® and identified SAGE-547 metabolite plasma concentrations;
3. QT/QTc interval changes and plasma concentrations of SAGE-547;
4. Number of days in the ICU, number of days in hospital, discharge destination from the ICU, discharge destination from the hospital, dischargeability criteria, resource use for subjects discharged from hospital before Visit 12/12R;

### 5. Full Outline of UnResponsiveness (FOUR) Score;

### 6. Glasgow Outcome Score (GOS);

### 7. Supervision Rating Scale (SRS);

8. Modified Rankin Score (mRS) (age ≥17 years).

### Inclusion Criteria

The following inclusion criteria must be met for individuals to be eligible for the trial.

1. Subjects two (2) years of age and older.
2. Subjects who have:
Failed to respond to the administration of at least one first-line agent (e.g., benzodiazepine or other emergent initial AED treatment), according to institution standard of care, and;
Failed to respond to at least one second-line agent (e.g., phenytoin, fosphenytoin, valproate, phenobarbital, levetiracetam or other urgent control AED), according to institution standard of care, and;
Not previously been administered a third-line agent but have been admitted to an intensive care unit with the intent of administering at least one third-line agent for at least 24 hours; or who have previously failed zero, one or more wean attempts from third-line agents and are now on continuous intravenous infusions of one or more third-line agent and in an EEG burst or seizure suppression pattern; or who have previously failed one or more wean attempts from third-line agents and are now either not on a continuous intravenous infusion of at least one third-line agent or are on a continuous intravenous infusion of one or more third-line agent but not in an EEG burst or seizure suppression pattern.

### Exclusion Criteria

None of the following exclusion criteria can be met for individuals to be eligible for the trial.

1. Subjects who are pregnant.
2. Subjects with a known allergy to progesterone or allopregnanolone or any excipients in SAGE-547.
3. Subjects with SRSE due to anoxic/hypoxic encephalopathy with highly malignant/malignant EEG features (Westhall, Rosetti et al. 2016).
4. Children (subjects aged less than 17 years) with an encephalopathy due to a rapidly progressing underlying neurological disorder.
5. Subjects who have any of the following:
a. a GFR low enough to warrant dialysis but for whatever reason, but dialysis that would adequately remove Captisol® is not planned;
b. severe cardiogenic or vasodilatory shock requiring two or more pressors that is not related to third-line agent use;
c. fulminant hepatic failure;
d. no reasonable expectation of recovery (for instance, a likely outcome is persistent vegetative state) or life-expectancy, in the opinion of the investigator, of less than 30 days.
6. Subjects who are being administered more than three third-line agents concomitantly or in whom the qualifying wean cannot be completed within 24 hours, or who are being administered a thirdline agent for other indications such as management of raised intra-cranial pressure that would preclude weaning according to this protocol.
7. Subjects with a living will that does not allow heroic measures.
8. Subjects who have been exposed to an investigational medication or device within 30 days; the exception to this is that participation in the Established Status Epilepticus Treatment Trial or ESETT within 30 days of screening for the 547-SSE-301 trial is allowed. 9. Subjects who have been treated or randomized in this trial or any other trial employing SAGE-547 previously (i.e., subjects may not have received study drug/placebo and then re-enroll).

### Pharmacogenetic Research

In addition to fulfilling all of the selection criteria described above, for inclusion in genetic research, subjects will also need to provide specific informed consent for genetic sampling and analyses, not have received a non-leukocyte-depleted transfusion within 120 days and not have had a previous allogenic bone marrow transplant.

### Statistical Analysis

A separate SAP will provide a detailed description of the analyses to be performed in the study. The SAP will be finalized and approved prior to database lock. Any deviations from or changes to the SAP following database lock will be described in detail in the final clinical study report.

Analysis of Primary Endpoint The analysis of response to treatment between SAGE-547 treated subjects and placebo treated subjects will be performed using a Cochran-Mantel-Haenszel test with variables for treatment, concomitant pentobarbital/thiopental use (yes or no) and number of previous third-line agent wean attempts prior to randomization (one vs two or more). In this analysis, a treatment responder is a subject who is successfully weaned off all third-line agents and blinded study medication prior to the end of study treatment infusion and remains off third-line agents for at least 24 hours following cessation of the blinded study medication, and who has concurrent signs of physiologic brain activity as determined by EEG. The analysis will be based on the ITT Population. The comparison of treatment response rates will be conducted at the 5% level of significance.

### Analysis of Secondary Efficacy Endpoints

The primary analysis will be repeated for the MITT population. Secondary endpoints will be compared between SAGE-547 and placebo treated subjects in the ITT population with a hierarchical testing process at the 5% level of significance. The analysis of secondary efficacy endpoints will use the order of endpoints as listed in the Endpoints Section. Assuming there is a significant difference between groups in the primary endpoint, the first secondary endpoint will be compared between groups. The testing process will continue until all endpoints have been evaluated or one of the analyses yields a non-significant result. Secondary analyses will be repeated for the MITT population. Summary statistics will be provided for all endpoints. Categorical endpoints (change from Baseline in CGI, secondary response rates, number of episodes of status epilepticus and proportion of new diagnoses of epilepsy) will be evaluated via Cochran-Mantel-Haenszel analysis with variables for treatment, concomitant pentobarbital/thiopental use and number of previous wean attempts. Analysis of continuous endpoints (time to re-institution of thirdline agents for primary and secondary response, number of days without status epilepticus or days without seizures) will be performed using Analysis of Variance with variables for treatment, concomitant pentobarbital/thiopental use and number of previous wean attempts. If it is found there are too many tied observations in the continuous data, a non-parametric analysis may also be performed. Summaries of safety endpoints will be based on the overall Safety Population which is defined as all subjects who at least initiated the infusion with blinded study treatment. Summary statistics will be calculated for primary and secondary response and duration of response for those subjects are treated with a higher dose of SAGE-547. Separate summaries will be derived for subjects initially receiving SAGE-547 and those initially receiving placebo. Summary statistics will be presented for subjects who were QW successes.

### Sample Size

The sample size of this study is based on the assumption of a 65% response rate to SAGE-547 treatment and a 35% response rate to placebo treatment and a 1:1 randomization schedule. Under these assumptions, with 70 subjects randomized to SAGE-547 and 70 subjects randomized to placebo, there would be >90% power for detecting a significant difference between groups at a 5% level of significance. Randomization will be stratified by concomitant pentobarbital/thiopental use (yes or no) and number of previous thirdline agent wean attempts prior to randomization (one vs two or more).

### Treatment of Subjects

### Dosing Schedule (Blinded Infusions)

SAGE-547 injection or placebo will be administered according to the dosing schedules in Table 1. The infusion rates to accomplish these μg/kg/h doses will be calculated according to the weight of the subject obtained within the eight-hour window that follows the declaration of the QW failure at V2 and prior to dosing at V3. The infusion rates will be applied to blinded study drug for the first infusion of study medication.

TABLE 1
SAGE-547 or Placebo Dosing Schedule

| Hour | Type and Duration of Study Drug Infusion | Description |
|---|---|---|
| H 1 | One hour loading Infusion | 300 μg/kg/h |
| H 2-H 120 | 119 hour maintenance infusion | 90 μg/kg/h |
| H 121-H 128 | 8 hour taper | 65 μg/kg/h |
| H 129-H 136 | 8 hour taper | 45 μg/kg/h |
| H 137-H 144 | 8 hour taper | 25 μg/kg/h |

### Dosing Schedule (Open-Label Infusions)

Those subjects that qualify for the open-label study drug will be administered SAGE-547 injection according to the dosing schedule in Table 2. The dose will be administered on a μg/kg/h basis, and the subject's weight measured prior to dosing during V10 will be used to calculate the doses. These infusion rates will be applied to SAGE-547 treatment for the open-label second infusions of study medication.

TABLE 2
SAGE-547 Open-Label Dosing Schedule

| Hour | Type and Duration of SAGE-547 Infusion | Description |
|---|---|---|
| H 1 | One hour loading Infusion | 300 μg/kg/h |
| H 2-H 120 | 119 hour maintenance infusion | 150 μg/kg/h |
| H 121-H 126 | 6 hour taper | 125 μg/kg/h |
| H 127-H 132 | 6 hour taper | 95 μg/kg/h |
| H 133-H 138 | 6 hour taper | 65 μg/kg/h |
| H 139-H 144 | 6 hour taper | 35 μg/kg/h |

### Route of Administration

SAGE-547 injection is intended for IV administration only. The compatibility of SAGE-547 with other drugs is unknown, and the product should be administered via a dedicated peripheral IV line using any Sage-supplied study-specific IV administration bags and lines. In order to preserve blinding, these instructions also apply to the blinded placebo infusions.

### Treatment Period

The treatment period with double-blind SAGE-547 or placebo is six 24-hour periods (144 hours), which may encompass 6-7 calendar days. The open-label study drug treatment period is of identical duration.

### Concomitant Medications, Procedures and Treatments

Subjects will receive the standard of care for SRSE. Any concomitant medication, procedure, and treatment determined necessary for the welfare of the subject may be given at the discretion of the Investigator at any time during the study. For all randomized and the first 50 qualifying wean success (QWS) subjects, detailed information on all concomitant medications (concomitant AEDs, third-line agents, pressors, and other concomitant medications), procedures, and treatments should be documented throughout the study from Screening through Visit 12/12R, as described below, and recorded on the eCRF. For subsequent QWS subjects, after the detailed information on all concomitant AEDs, pressors, third-line agents, procedures, and treatments collected during the

screening period (Visits 1 and 2) has been recorded, only minimal information (to include the name of the concomitant AED or pressor, third-line agent, procedure, and/or treatment, and the start and stop dates and times) from Visit 3 through Visit 12 will be recorded in the eCRF. For subsequent QWS subjects, after the detailed information on all concomitant medications collected during the screening period (Visits 1 and 2) has been recorded, only minimal information, to include the name of the concomitant medications and indication only will be recorded from Visit 3 through Visit 12. SAGE-547 has demonstrated inhibitory effects on cytochrome P450 isoenzyme 2C9 (CYP2C9). Therefore, AEDs such as phenytoin and phenobarbital that are primarily metabolized by CYP2C9 should be closely monitored during SAGE-547 administration to ensure target exposures are maintained.

### Concomitant AEDs

Subjects may enter the study on AEDs, and be administered AEDs during the study. All of these must be recorded on the eCRF with details including date and time of starting and stopping each AED, the route of administration, changes in doses, and frequency of administration. In addition, details of the first-line and second-line agents used to treat the subject prior to consenting for the study will be recorded on the eCRF, noting at what point the subject was deemed to have “failed first-line agents” and “failed second-line agents”.

### Concomitant Third-Line Agents

All third-line agents administered since the diagnosis of SE will be recorded on the eCRF, whatever path the patient took for entry into the study. Details will include the name of the drug, the start and stop dates and times, and, after consent, the doses with start and stop times for each change in dose. This recording of third-line agents will continue throughout the study until Visit 12/12R. In addition, those changes in dose that constitute a wean attempt will be marked as such on the eCRF. In this way the start and stop dates and times of all wean attempts will be recorded, as will the type of wean (QW, OW, or TW) and the outcome of the wean attempt (successful, failed and dose of this third-line agent increased or failed and this thirdline agent stopped and another third-line agent started). Those wean attempts that occur after the TW up to and including Visit 12/12R will be marked “AW”. Some third-line agents (such as propofol) are also used to induce sedation to facilitate a subject's ability to tolerate the ventilator. For subjects requiring such sedation, propofol is the preferred agent unless there are contraindications. The eCRF will make clear from the indication for the third-line agent when it is being used for burst or seizure suppression and when it is being used as ventilator support or for another purpose. The investigator will add a note to the eCRF to justify that the doses used are appropriate for sedation and not for seizure or burst suppression. The doses used for ventilator support and other similar uses are usually much lower than those used for seizure or burst suppression, and use of these third-line agents for these other purposes does not constitute a wean failure under this protocol.

### Concomitant Pressors

The start and stop dates and times of all pressors will be recorded as well as the start and stop dates and times of all dose changes.

### Other Concomitant Medications

All other concomitant medications must be recorded on the eCRF with details including date and time of starting and stopping, the route of administration, dose, and frequency of administration.

### Example 3: Response of Patients with SRSE to Treatment with SAGE-547 Injection where the SRSE is Preceded by a Structural Modification in the Brain

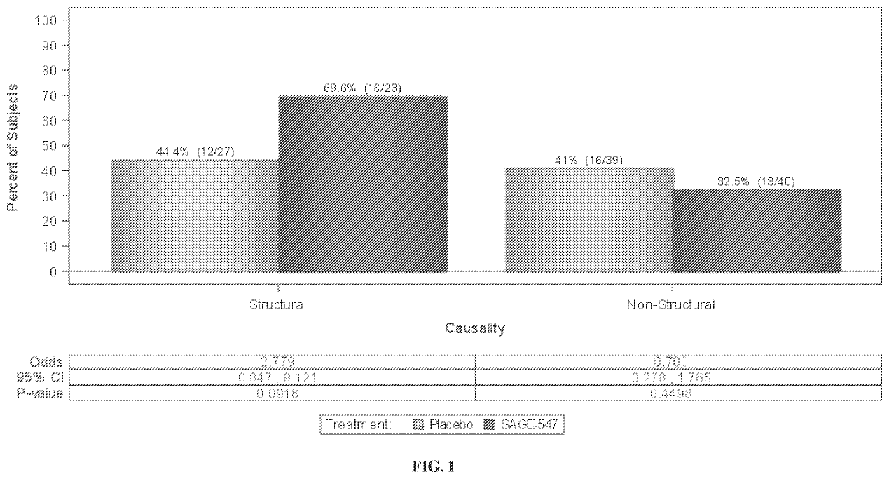

In accordance with the trial protocal in Example 2 described herein, the amount of treatment responders with SAGE-547 injection relative to placebo where the SRSE is preceded by a structural modification in the brain is shown in FIG. 1. Briefly, 12 of 27 patients who had a structural modification in the brain responded to treatment by placebo, while 16 of 23 patients who had a structural modification in the brain responded to treatment by SAGE-547 injection.

Additionally, the amount of treatment responders with SAGE-547 injection relative to placebo where the SRSE was not preceded by a structural modification in the brain is also shown in FIG. 1. 16 of 39 patients who did not have a structural modification in the brain responded to treatment by placebo, while 13 of 40 patients who did not have a structural modification in the brain responded to treatment by SAGE-547 injection.

### Example 4: Response of Patients with SRSE to Treatment with SAGE-547 Injection where the SRSE is Correlated by Etiology

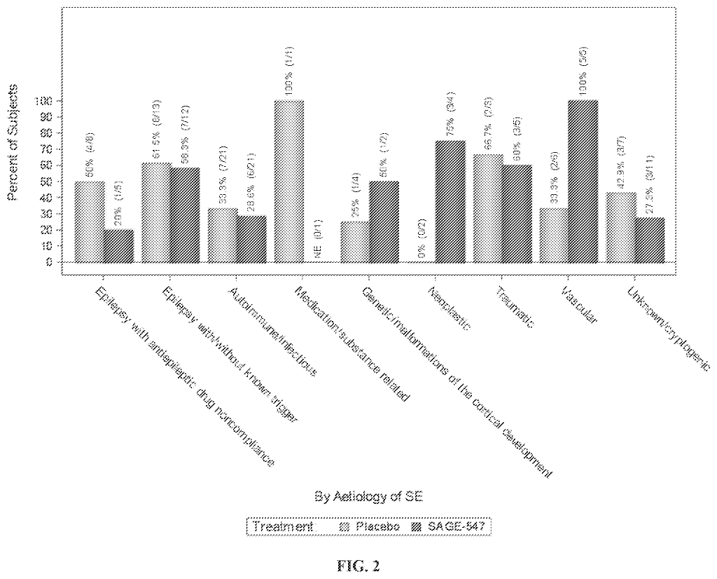

In accordance with the trial protocal in Example 2 described herein, the amount of treatment responders with SAGE-547 injection relative to placebo where the SRSE is correlated by etiology modification in the brain is shown in FIG. 2. Exemplary etiologies include epilepsy with antiepileptic drug noncompliance, epilepsy with or without a known trigger, autoimmune or infectious etiologies, medication or substance-related etiologies, genetic etiologies or malformation of the cortical development, neoplastic etiologies, traumatic etiologies, vascular etiologies, and unknown or cryptogenic etiologies.

1. A method of treating a subject having a seizure-related disorder, wherein said seizure-related disorder is preceded by a condition related to a structural modification in the brain of said subject, the method comprising: administering to said subject, an effective amount of allopregnanolone, thereby treating said subject,

2. A method of treating a subject having a seizure-related disorder, wherein said seizure-related disorder is preceded by a condition selected from the group consisting of a brain aneurysm with associated hemorrhage, an intraparenchymal hemorrhage, a brain arteriovenous malformation with associated hemorrhage, an ischemic stroke, a focal cortical dysplasias or malformation of cortical development, an intraparenchymal brain tumor, post-traumatic porenenchephaly or gliosis, a cerebral abscess, a central nervous system infection, encephalitis, multiple sclerosis, and a demyelinating lesion, the method comprising: administering to said subject, an effective amount of allopregnanolone, thereby treating said subject.

3. The method of claim 1, wherein, concurrent with said administering, said subject is under general anesthesia.

4. The method of claim 1, the method comprising:
administering a first dose,
administering a second dose; and
administering a third dose,
said allopregnanolone doses being sufficient to treat said subject.

5-8. (canceled)

9. The method of claim 12, wherein the second dose is administered over a period of time that is at least 2, 3, 4, 5, 6 times longer in duration than that of said third dose.

10. (canceled)

11. (canceled)

12. The method of claim 1, wherein one, two or all of said doses are injected.

13.-15. (canceled)

16. The method of claim 1, wherein said anesthetic is selected from benzodiazepine, propofol, pentobarbital, and ketamine.

17.-58. (canceled)

59. The method of claim 1, said first dose is administered at a dosage rate of 200-3500 μg/kg/hour.

60. The method of claim 1, said first dose is administered at a dosage rate of 200-350 μg/kg/hour, 250-300 μg/kg/hour, 280-290 μg/kg/hour, 286 μg/kg/hour, 287 μg/kg/hour, or 288 μg/kg/hour.

61.-70. (canceled)

71. The method of claim 1, wherein said second dose is administered for a period of time that is between 48 and 192 hours, 60 and 144 hours, 60 and 120 hours, 80 and 110 hours, or 90 and 100 hours.

72.-91. (canceled)

92. The method of claim 1, said second dose is administered at an amount of allopregnanolone/unit time of 25-1500 μg/kg/hour.

93. The method of claim 1, said second dose is administered at an amount of allopregnanolone/unit time of 25-150 μg/kg/hour, 50-100 μg/kg/hour, 75-100 μg/kg/hour, 85 μg/kg/hour, 86 μg/kg/hour, or 87 μg/kg/hour.

94.-118. (canceled)

119. The method of claim 1, wherein the allopregnanolone is provided in a composition comprising a cyclodextrin.

120. The method of claim 1, wherein the allopregnanolone is provided at a concentration of 0.1 to 10 mg/mL allopregnanolone.

121.-125. (canceled)

126. The method of claim 119, wherein the cyclodextrin is present in the composition at 1-30%, 2-18%, 10-15% by weight of cyclodextrin per volume of composition.

127. The method of claim 119, wherein the cyclodextrin is present in the composition at 1, 2.5, 5, 10, 12, 13, 15, 30% by weight of cyclodextrin per volume of composition.

128. (canceled)

129. The method of claim 119, wherein the cyclodextrin is present in the composition at 1-30%, 2-18%, 10-15% by weight of cyclodextrin per volume of composition and the allopregnanolone is provided at a concentration of 0.1, 0.5, 1, 1.25, 2.5, 3.75, 5, 6.25, 7.5, 8, 9, or 10 mg/mL allopregnanolone.

130. The method of claim 119, wherein the cyclodextrin is present in the composition at 1, 2.5, 5, 10, 12, 13, 15, 30% by weight of cyclodextrin per volume of composition and the allopregnanolone is provided at a concentration of 0.1, 0.5, 1, 1.25, 2.5, 3.75, 5, 6.25, 7.5, 8, 9, or 10 mg/mL allopregnanolone.

131.-141. (canceled)

142. A method of treating a subject having, status epilepticus (SE), refractory status epilepticus (RSE) or super-refractory status epilepticus (SRSE), comprising:
administering a first/load, wherein administration of said first dose: begins 2-120 hours after induction of general anesthesia; lasts for 30-90 minutes; and results in a plasma level of allopregnanolone of 100-2000 nM allopregnanolone;
administering a second/maintenance dose, wherein, the administration of said second dose begins not longer than 1-60 minutes after the end of the second dose; lasts for 1-6 days; and results in a plasma level of allopregnanolone of 100-2000 nM allopregnanolone;
administering a third downward taper dose, wherein, the administration of said third downward taper dose begins not longer than 1-60 minutes after the end of the third dose; lasts for 10-100 hours; and results in a plasma level of allopregnanolone of 0-1500 nM allopregnanolone;
wherein, collectively, the administrations are provided in sufficient amount to treat said subject, wherein said seizure-related disorder is preceded by a condition related to a structural modification in the brain of said subject.

143.-146. (canceled)

147. The method of claim 142, further comprising,
administering an amount of a composition selected from benzodiazepines, propofol, and barbiturates, sufficient to place said subject under general anesthesia;

148.-168. (canceled)
